(19) United States
(12) Patent Application Publication
(10) Pub. No.: US 2023/0028019 A1
(43) Pub. Date: Jan. 26, 2023

(54) BONDED NEUROTOXINS

(71) Applicant: THE UNIVERSITY OF SHEFFIELD, Sheffield, South Yorkshire (GB)

(72) Inventors: Bazbek DAVLETOV, Sheffield (GB); Charlotte Michelle LEESE, Sheffield (GB)

(21) Appl. No.: 17/756,480

(22) PCT Filed: Nov. 25, 2020
(86) PCT No.: PCT/GB2020/052991

§ 371 Date: May 26, 2022

(30) Foreign Application Priority Data
Nov. 27, 2019 (GB) 1917265.9

(51) Int. Cl. C07K 14/33; C07K 14/705; C12N 9/52

(52) U.S. Cl. CPC C07K 14/33; C07K 14/705; C12N 9/52; C12Y 304/24069

(57) ABSTRACT

The present invention provides novel neurotoxins and compositions comprising the same. The neurotoxins are useful in therapy, particularly for preventing, regulating or reducing neuropathic pain or sweating. Methods and kits for producing the neurotoxins are also provided.

The present invention provides novel neurotoxins and compositions comprising the same. The neurotoxins are useful in therapy, particularly for preventing, regulating or reducing neuropathic pain or sweating. Methods and kits for producing the neurotoxins are also provided.

### BACKGROUND

The closely related Botulinum neurotoxin family consists of seven primary distinct botulinum neurotoxins (Bots; types A to G), which cause human and animal botulism [1]. Botulinum neurotoxin types A, B, E and F have been shown to cause disease in humans, with types A, B and E being associated with foodborne illness. Botulinum neurotoxins type C and type D cause botulism in birds and mammals. No disease has currently been attributed to botulinum neurotoxin type G. There are also many chimeric botulinum neurotoxins found in nature which are made in a mosaic fashion from the primary botulinum neurotoxins and the total number of putative botulinum neurotoxins is over 250 (summarised in [2]).

Each botulinum neurotoxin is synthesized as a ˜150 kDa single chain protein with three structurally independent domains: a SNARE peptidase domain, a translocation domain, and a neuronal binding domain (FIG. 1A; [3]). In vivo, the single chain protein is subsequently cleaved by Clostridial or host proteases to generate an N-terminal ˜50 kDa enzymatic Light chain (L; comprising the SNARE peptidase domain) and a ˜100 kDa Heavy chain (H; comprising the translocation domain—Hn, and the C-terminal neuronal binding domain—Hc) (FIG. 1A). The Light and Heavy chains remain attached via a single disulphide bond, a peptide loop and further non-covalent interactions.

The three protein domains of a botulinum neurotoxin perform distinct roles in toxin delivery and activity within host cells. The neuronal binding domain (NBD also known as Hc) is required to specifically bind to target host cells (neurons) and enter recycling vesicles; the translocation domain (Hn) facilitates transition of the toxic light chain (L, also known as SNARE peptidase) from a vesicle into the cytosol of the host cell where the peptidase domain catalyses the proteolysis of one of three soluble N-ethylmaleimide-sensitive fusion protein attachment receptors (SNAREs) in the cell, namely VAMP/synaptobrevin, a synaptosome-associated protein of 25 kDa (SNAP25), or syntaxin. As these substrate proteins are essential components of the host vesicular membrane fusion apparatus, cleavage of any one of these proteins blocks neurotransmitter release from the host cell.

Botulinum neurotoxins (also referred to as “Bots” herein) are important biopharmaceuticals for treatment of various muscular and secretory conditions [4]. These toxins silence neuromuscular junctions and also can block neurotransmitter release from many types of neurons. Botulinum neurotoxin type A (botulinum type A; or Bot/A) is also used in cosmetic medicine [4]. Injections of Bot/A cause local nerve block leading to lasting muscle relaxation up to five months [4]. Bot/A has therefore proven to be of great medical importance due to its ability to cause a very long neuromuscular paralysis upon local injections of minute amounts (picogram doses) [4].

Practically every part of the human body including the brain [Botulinum toxin therapy for neuropsychiatric disorders, US 20040180061 A1] can be treated using Bot/A. An example of a form of Bot/A that has been used successfully for cosmetic treatment is Botox®. Since the paralysis of neuromuscular junctions is reversible, the sustained relaxation of muscles requires repeat injections every three to four months. Bot/A can block innervation of not only striated muscles but also of smooth muscles. Furthermore, the cholinergic junctions of the autonomous nervous system that control sweating, salivation and other types of secretion are as sensitive to Bot/A and Bot/B as are the neuromuscular junctions. Therefore, botulinum-based treatments have recently expanded to include a dazzling array of nearly a hundred conditions from dystonias to gastrointestinal and urinary disorders.

The effectiveness of Bot/A in clinical medicine has led to increasing interest in other members of the botulinum family. Comparative studies have demonstrated that Bot/A has the longest paralysing effect among the seven immunologically distinct serotypes of botulinum neurotoxin (A-G), thus underpinning the usefulness of specifically Bot/A in the treatment of neurological disorders.

The extreme toxicity of botulinum neurotoxins indicates that the peripheral nerve endings carry molecules that can serve as botulinum neurotoxins' high-affinity receptors. Indeed, several synaptic vesicle proteins have been shown to act as receptors for botulinum neurotoxins. While the heavy chains are responsible for botulinum neurotoxins' binding to nerve terminals, the light chains are potent endopeptidases that attack the vesicle fusion machinery and therefore have to get inside the nerve terminal. Botulinum neurotoxins accomplish this task by hijacking the vesicle endocytosis route. As the pH of the recycling vesicle's interior drops, the botulinum neurotoxins undergo major conformational changes. This enables the translocating domain (known as Hn) of the heavy chains to form putative channels across the vesicular membrane through which the partially unfolded light chains slip into the cytosol. On entry into the cytosol, reduction of the disulphide bond frees the light chain from the heavy chain.

Botulinum neurotoxin light chains are potent SNARE peptidases that attack a number of isoforms of the three SNARE proteins that mediate vesicle fusion and therefore neurotransmitter release. It is now known that Bot/A and Bot/E proteolyse SNAP-25, while botulinum neurotoxins B, D, F and G cleave VAMP on the synaptic vesicles. SNAP-25 shortened by only nine amino acids by Bot/A retains its ability to interact with the plasma membrane syntaxin and vesicular synaptobrevin but cannot mediate the normal vesicle fusion process. Further information about botulinum neurotoxins can be found in [5-11]. The complete sequence information for Bot/A was published in [12].

Biopharmaceutical production of Bot/A, dubbed the most toxic toxin known to humans, requires great care and is strictly regulated. There are only a dozen companies worldwide that are authorised to produce Bot/A as a biopharmaceutical medicine.

A protein stapling technique has previously been used to produce a lesser paralysing Bot/A [13, 14]. This method requires production of two parts of Bot/A—LHn and Hc, each fused to a complementary SNARE-based linker. The two parts of Bot/A are spontaneously recombined on addition of a ‘stapling’ peptide. The stapling system located between the two Bot/A parts causes an elongation of Bot/A which may account for impeded entry into motor neurons causing a decrease in paralysis. The ‘stapled’ Bot/A has been shown to be effective in pain alleviation at 200 nanogram amounts in rodents due to penetration into sensory neurons possibly via large vesicle recycling [15]. Recently, potential botulinum neurotoxin production from two modified botulinum parts using a sortase catalytic reaction has been also demonstrated [16]. Similar to ‘stapling’, the sortase approach requires addition of a third element (the sortase) to facilitate formation of functional botulinum neurotoxin from two parts. However, sortase reactions are reversible and formation of functional botulinum neurotoxin complex is therefore be transient. Furthermore, research using sortase for protein modification has reported poor reaction rates and the requirement for Ca²⁺ in the reaction. Some progress has been made in circumventing reaction reversibility, which require selective removal or deactivation of transpeptidation products. Such strategies include running the reaction under dialysis conditions to separate out low molecular weight by-products or the use of β-hairpins or depsipeptides, or masked metal binding peptides that prevent transpeptidation products from re-entering the catalytic cycle. Such strategies are complex and can be inconsistent.

There is a need for improved methods for generating novel neuronal modulators in a safe manner.

### BRIEF SUMMARY OF THE DISCLOSURE

The inventors have used a Spytag peptide and cognate Spycatcher protein conjugation system to make a novel functional botulinum neuronal modulator (referred to as “bonded botulinum” or “BonBot” herein) from two independently produced non-functional parts (FIG. 1A). In doing so, they have developed a system for safely manufacturing a neurotoxin from two parts only, without addition of any other catalysts or staples.

Spycatcher technology is a newly introduced protein bonding technique, which allows covalent linking of proteins (1); see also WO2011098772 and WO2018197854. This bonding technique relies on formation of an isopeptide bond between a protein called Spycatcher (15 kDa) and a 12 amino acid peptide called Spytag. This covalent peptide interaction is a simple and powerful tool for bioconjugation. Fusion of the Spycatcher and Spytag to two independent proteins allows bonding of these proteins by simple mixing. The inventors have surprisingly shown that the Spycatcher:Spytag technology can be used to effectively covalently link two relatively large independent proteins (approximately 100 KDa and 50 KDa respectively) to generate a functional neurotoxin.

Although Spycatcher technology has been used to exemplify the invention, other techniques for covalently linking two independently produced non-functional parts of a botulinum neurotoxin may also be used. For example, other bipartite bonding systems can be used to produce binary botulinum molecules, including for example split CnaB, RrgA and other similar protein domains. More details of these alternative technologies are provided below.

The inventors have shown that BonBot/A has a slightly reduced SNAP25-cleaving potency compared with native Bot/A (see SNAP25 data shown in FIG. 10 for example). BonBot/A was also functional in causing muscle paralysis but at a higher dose compared to native Bot/A (FIG. 2). The novel constructs with reduced paralytic properties could advantageously be used to treat neuropathic pain with minimal adverse effect on muscle function. The inventors have investigated this further using a BonBot/A construct with a novel extended rigid linker (referred to as a rigid trihelical extension herein) inserted between the translocation domain and the neuronal binding domain (ext-BonBot/A, FIG. 3A). Surprisingly, ext-BonBot/A showed high efficiency in treating neuropathic pain with minimal adverse effect on muscle function. No muscle paralysis was detected even after injection of 100 ng ext-BonBot/A. BonBot/A and ext-BonBot/A are therefore particularly useful in preventing, reducing and/or treating neuropathic pain or other non-muscle related conditions, such as excessive sweating.

BonBot/A is larger than any other protein that have been linked by the isopeptide pairing method, and therefore it is not obvious that it would be possible to add further protein elements. The efficient ‘large protein’ linking process is further exemplified by duplication of botulinum parts. Specifically, an example is presented where LHn/A carrying two Spycatcher molecules fused linearly allows production of a new botulinum molecule with two binding domains upon simple addition of Spytag-Hc/A (FIG. 9). Functionally, such ‘multiple binding domains’ botulinum molecule possesses an enhanced ability to cleave SNAP25 (FIG. 9) and therefore is expected to have stronger physiological effects since the cleavage of SNAP25 is the basis of botulinum neurotoxin type A function [4]. The beneficial effects of constructs with multiple binding domains have been demonstrated previously. For example, duplication of clostridial neuron-binding domains results in increased delivery of enzymatic and imaging cargoes into neurons [17]. Duplication of binding domains could be of homologous or heterologous nature, where binding domains can be coupled from different botulinum serotypes. Such enhanced botulinum therapeutics should achieve therapeutic effects with lower doses compared to the current doses of these bacterially-derived immunogenic molecules, especially where high doses are required, for example in the cases of severe migraine and cerebral palsy. Such enhanced botulinum therapeutics exhibit faster therapeutic effects [17].

Although the invention has been demonstrated using Bot/A domains, the inventive concept applies equally to neurotoxins generated from other closely related botulinum SNARE peptidase, translocation and neuronal binding domains, and chimeras thereof. The general applicability of the invention has been demonstrated by the inventors in FIG. 4, which demonstrates the invention with a functional neurotoxin chimera (referred to as BonBot/C herein) generated from the SNARE peptidase and translocation domains of Bot/A and the neuronal binding domain of Bot/C. By selecting specific neuronal binding domains, the resultant neurotoxin can be used to more effectively target specific neuronal populations.

The methods described herein allow the user to work with, optimise and modify atoxic, harmless parts of botulinum toxins, which can subsequently be joined together to form the functional toxin (including neurotoxin chimeras). The techniques described herein can therefore advantageously be used to make modified neurotoxins, thereby facilitating the production of tailored nerve blockers for future medical applications.

In one aspect, the invention provides a neurotoxin comprising a SNARE peptidase domain, a translocation domain and a neuronal binding domain, wherein two of the domains are present within a disulphide-linked polypeptide that is covalently linked to the third domain via an isopeptide bond.

Suitably, the SNARE peptidase domain, translocation domain and neuronal binding domain may be botulinum neurotoxin SNARE peptidase, translocation and neuronal binding domains. Suitably, the SNARE peptidase domain, translocation domain and neuronal binding domain may be botulinum neurotoxin type A SNARE peptidase, translocation and neuronal binding domains.

Suitably, the SNARE peptidase domain and the translocation domain may be present within the disulphide-linked polypeptide that is covalently linked to the neuronal binding domain via an isopeptide bond. Optionally, the disulphide-linked polypeptide may further comprise a rigid trihelical extension.

Suitably, the isopeptide bond may be formed between a peptide and its cognate protein, wherein:

a) the peptide is attached to the disulphide-linked polypeptide and the cognate protein is attached to the third domain; or

b) the cognate protein attached to the disulphide-linked polypeptide and the peptide is attached to the third domain.

Suitably, the peptide may comprise an amino acid sequence of SEQ ID NO:1 and the cognate protein may comprise an amino acid sequence of SEQ ID NO:2.

Suitably, the neurotoxin may comprise:

a) a disulphide-linked polypeptide comprising the SNARE peptidase domain, the translocation domain and a C-terminal cognate protein comprising the amino acid sequence of SEQ ID NO: 2; and

b) the neuronal binding domain attached to an N-terminal peptide comprising the amino acid sequence of SEQ ID NO:1,

wherein the disulphide-linked polypeptide is covalently linked to the neuronal binding domain via an isopeptide bond between the cognate protein of a) and peptide of b).

Suitably, the SNARE peptidase domain may comprise an amino acid sequence having at least 80% identity to the amino acid sequence of SEQ ID NO: 3 and the translocation domain may comprise the amino acid sequence of SEQ ID NO:4 or SEQ ID NO: 5.

Suitably, the neuronal binding domain may comprise the amino acid sequence of SEQ ID NO: 6 or SEQ ID NO:7.

In any embodiment described herein, the disulphide-linked polypeptide may typically comprise the SNARE peptidase domain and the translocation domain, which is then covalently linked to the neuronal binding domain via an isopeptide bond. Typically, in such embodiments, the SNARE peptidase domain is N-terminal to the translocation domain within the disulphide-linked polypeptide (such that the translocation domain is C-terminal to the SNARE peptidase domain). Typically, the disulphide-linked polypeptide is then N-terminal to the neuronal binding domain in the covalently linked neurotoxin. In such constructs, the covalent link is therefore between the translocation domain of the disulphide-linked polypeptide and the neuronal binding domain. Preferably, the covalent link spatially separates the translocation domain from the neuronal binding domain by a suitable distance e.g. by a distance of at least 4 nm e.g. at least 4.7 nm. Suitably, this distance may be further increased by using an extension sequence such as a spacer domain, which is described elsewhere herein. Inclusion of spacer domains may increase the distance between the translocation domain and the neuronal binding domain to, for example, at least 5 nm, at least 5.5 nm, at least 5.6 nm, at least 10 nm e.g. at least 10.3 nm etc. Increasing the distance between the translocation domain and the neuronal binding domain is shown herein to be beneficial in preventing, regulating or reducing neuropathic pain and sweating.

In any embodiment described herein, the neurotoxin may comprise more than one neuronal binding domain. As exemplified herein, the neurotoxin may comprise two neuronal binding domains (see for example, BonBot/AA described herein). Suitably, the disulphide-linked polypeptide may comprise the SNARE peptidase domain and the translocation domain, which is then covalently linked to the neuronal binding domains via isopeptide bonds. Typically, in such embodiments, the SNARE peptidase domain is N-terminal to the translocation domain within the disulphide-linked polypeptide (such that the translocation domain is C-terminal to the SNARE peptidase domain). Typically, the disulphide-linked polypeptide is then N-terminal to the neuronal binding domains in the covalently linked, i.e. bonded, neurotoxin. In such constructs, one or more post-translational covalent links is therefore between the translocation domain of the disulphide-linked polypeptide and each of the neuronal binding domains. The covalent link(s) then spatially separate the translocation domain from each of the neuronal binding domains by a suitable distance e.g. by a distance of at least 4 nm e.g. at least 4.7 nm. Suitably, this distance may be further increased by using an extension sequence, such as a rigid trihelical extension. Inclusion of such sequences may increase the distance between the translocation domain and each of the neuronal binding domains to, for example, at least 5 nm, at least 5.5 nm, at least 5.6 nm, at least 10 nm, e.g. at least 10.3 nm etc. In another aspect, the invention provides a composition comprising a neurotoxin of the invention, and a pharmaceutically acceptable excipient, adjuvant, diluent or carrier.

In another aspect, the invention provides the use of a composition of the invention for therapy.

Suitably, the composition may be for preventing, regulating or reducing neuropathic pain or sweating in a subject.

In another aspect, the compositions of the invention may be for use in therapy.

Suitably, the compositions may be for use in preventing, regulating or reducing neuropathic pain or sweating in a subject.

In another aspect, the invention provides a therapeutic method, the method comprising administering a composition of the invention to a subject.

In a further aspect, the invention provides a method of preventing, regulating or reducing neuropathic pain or sweating in a subject, the method comprising administering a composition of the invention to the subject.

In a further aspect, the invention provides a method of producing a neurotoxin comprising a SNARE peptidase domain, a translocation domain and a neuronal binding domain, the method comprising mixing a disulphide-linked polypeptide comprising two of the domains with a polypeptide comprising the third domain, wherein

(i) a peptide is attached to the disulphide-linked polypeptide and a cognate protein is attached to the third domain; or

(ii) a cognate protein attached to the disulphide-linked polypeptide and a peptide is attached to the third domain,

such that, on mixing, an isopeptide bond is formed between the peptide and its cognate protein to covalently link the disulphide-linked polypeptide to the third domain.

In another aspect, the invention provides a kit for producing a neurotoxin comprising a SNARE peptidase domain, a translocation domain and a neuronal binding domain, the kit comprising:

(a) a disulphide-linked polypeptide comprising two of the domains; and

(b) a polypeptide comprising the third domain, wherein
(i) a peptide is attached to the disulphide-linked polypeptide and a cognate protein is attached to the third domain; or
(ii) a cognate protein attached to the disulphide-linked polypeptide and a peptide is attached to the third domain,

such that, on mixing, an isopeptide bond is formed between the peptide and its cognate protein to covalently link the disulphide-linked polypeptide to the third domain.

Suitably, the SNARE peptidase domain, translocation domain and neuronal binding domain may be botulinum neurotoxin SNARE peptidase, translocation and neuronal binding domains.

Suitably, the SNARE peptidase domain, translocation domain and neuronal binding domain may be botulinum neurotoxin type A SNARE peptidase, translocation and neuronal binding domain.

Suitably, the peptide may comprise an amino acid sequence of SEQ ID NO:1 and the cognate protein may comprise an amino acid sequence of SEQ ID NO:2.

Suitably:

a) the disulphide-linked polypeptide may comprise the SNARE peptidase domain, the translocation domain and a C-terminal cognate protein comprising the amino acid sequence of SEQ ID NO: 2; and

b) the polypeptide may comprise the third domain comprises the neuronal binding domain attached to an N-terminal peptide comprising the amino acid sequence of SEQ ID NO:1,

wherein the disulphide-linked polypeptide is covalently linked to the neuronal binding domain via an isopeptide bond between the cognate protein of a) and peptide of b).

Suitably, the resultant neurotoxin may further comprise a rigid trihelical extension between the translocation domain and the neuronal binding domain. For example, the disulphide-linked polypeptide may further comprise the rigid trihelical extension.

Suitably, the SNARE peptidase domain may comprise an amino acid sequence having at least 80% identity to the amino acid sequence of SEQ ID NO: 3 and the translocation domain may comprise the amino acid sequence of SEQ ID NO:4 or SEQ ID NO: 5.

Suitably, the neuronal binding domain may comprise the amino acid sequence of SEQ ID NO: 6 or SEQ ID NO:7.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows formation of BonBot/A from two botulinum parts. A. Schematic showing the botulinum functional domains with Spycatcher and Spytag system inserted between LHn/A and Hc/A. B. Coomassie-stained SDS-PAGE gel shows formation of BonBot/A within 2 hours by simple mixing of LHn/A-Spycatcher and Spytag-Hc/A. Note the transition of LHn/A-Spycatcher into BonBot/A. C and D. An anti-SNAP25 immunoblot (C) and quantification (D) of the proportion of SNAP25 cleaved in differentiated SiMa neuroblastoma cells treated with BonBot/A for 48 hours is shown. E. An immunoblot shows that, in a negative control experiment, SNAP25 remains intact after 48 hrs in differentiated SiMa neuroblastoma cells treated with either 10 nM LHn/A-Spycatcher or 10 nM Spytag-Hc/A i.e. them being non-bonded.

FIG. 2 shows that BonBot/A paralyses gastrocnemius muscle following BonBot/A injections (black arrows), whereas its component parts do not. A. Images of rats injected with 20 ng of BonBot/A are shown. The injected leg was splayed and unable to support weight after 48 hrs. B. Images of rats injected with 100 ng of either LHn/A Spycatcher (left) or Spytag-Hc/A (right) are shown. No signs of muscle paralysis were observed when separate parts were injected.

FIG. 3 shows that the Spycatcher/Spytag system can be used to make enlarged botulinum molecules. A. A schematic showing the difference between BonBot/A and the extended (ext)BonBot/A. B. Coomassie-stained SDS-PAGE gel shows formation of the extended BonBot/A within 2 hrs upon mixing of LHn/A-extension-Spycatcher and Spytag-Hc/A. Note the transition of LHn/A-extension-Spycatcher into ext-BonBot/A. C and D. An anti-SNAP25 immunoblot (C) and quantification graph (D) showing the proportion of SNAP25 cleaved in differentiated SiMa neuroblastoma cells treated with ext-BonBot/A for 48 hours. E. Electromyographic measurements of Compound Muscle Action Potentials (CMAPs) in the rat gastrocnemius muscle following subcutaneous injections demonstrate that the extended BonBot/A at 2 ng causes minimal paralysis compared to the non-extended version (*=P<0.001, n=4; 2-way ANOVA, repeated measures, Tukey post hoc test).

FIG. 4 shows that the Spycatcher/Spytag system allows for formation of botulinum chimeras, such as BonBot/C. A. Coomassie-stained SDS-PAGE showing formation of BonBot/C within 2 hrs by mixing of LHn/A-Spycatcher and Spytag-Hc/C. Note the transition of LHn/A-Spycatcher into BonBot/C. B. Immunoblot showing the proportion of SNAP25 cleaved in differentiated SiMa neuroblastoma cells treated for 48 hrs with BonBot/C. C. Images of rats injected with 66 ng of BonBot/C are shown. The affected leg was splayed and unable to support weight.

FIG. 5 shows examples of purification of BonBot component parts. A. Coomassie-stained gel showing GST-fusion protein containing HcA with Spytag peptide with subsequent thrombin-induced release of Hc/A-Spytag and size-exclusion chromatography fractions containing purified Hc/A-Spytag. B. Coomassie-stained gel showing GST-fusion protein containing LHn/A with Spycatcher cognate protein with subsequent thrombin-induced release of LHn/A-Spycatcher and size-exclusion chromatography fractions containing purified LHn/A-Spycatcher. C. Coomassie-stained gel showing GST-fusion protein containing LHn/A, the rigid extension and Spycatcher cognate protein with subsequent thrombin-induced release of extended LHn/A-Spycatcher and size-exclusion chromatography fractions containing purified LHn/A-ext-Spycatcher.

FIG. 6 shows structural elements used in construction of BonBot molecules. A. The rigid trihelical spacer domain from syntaxin 1 protein used for engineering of the extended BonBot/A. Note, the trihelical structure allows addition of proteins specifically on opposite sides of the extension. Panels B and C show structural models of BonBot/A and Ext-BonBot/A respectively, with calculated sizes in nanometers. The models incorporate published structural elements from the RSCB PDB database with the following ID codes: 3BTA (LHn/A and Hc/A), 4MLI (Spytag+spycatcher) and 1EZ3 (syntaxin 1 trihelical domain).

FIG. 7. A and B. Changes in animal behaviour and gait were observed following SNI and sub-plantar injection of either vehicle or ext-BonBot/A. Animals injected with vehicle showed reluctance to place ipsilateral hindpaw onto the mesh surface and showed reduced weight bearing on the affected limb (A). In contrast, animals injected with ext-BonBot/A more readily made contact with the mesh flooring and showed more normal gait behavior (B). Images taken 7 days following SNI and 3 days following sub-plantar injection. C. Extended Bonded botulinum (ext-BonBot/A) reduces mechanical hypersensitivity in a rat model of neuropathic pain. Mechanical thresholds were measured in rats before and after spared nerve injury (SNI) surgery. Four days later, rats were injected with either vehicle or ext-BonBot/A (10 ng/30 microliters) into the injured rat paw (n=4 per group). Injections of ext-BonBot/A reversed mechanical hypersensitivity of the paw due to nerve injury. Data shows log of the mean of the 50% threshold±SEM. Statistical analysis was performed using GraphPad Prism v8.0 using repeated measures two-way ANOVA. P<0.05 was considered statistically significant.

FIG. 8 shows that intraplantar injection of ext-BonBot/A reduces the mechanical hypersensitivity in adult mice after nerve injury. Mechanical thresholds were assessed using Von Frey filaments before (b1, b2) and after nerve injury. Ext-BonBot/A (50 pg/20 microliter) was injected intraplantar on day 6 and recovery of mechanical sensitivity was followed until day 20. Data show means±SEM. *p<0.05. 50 pg ext-BonBot/A n=4; vehicle n=3.

FIG. 9 shows that a duplicated Spycatcher sequence fused to the LHn botulinum part can be used to assemble more efficient ‘double binding’ botulinum molecules. A. Schematic showing how single or duplicated Spycatcher domain within the same LHn/A protein can be used to bond either one or two copies of Spy-tagged binding domain, HcA. B. Coomassie stained SDS-PAGE gel showing the formation of bonded botulinum molecule carrying one or two copies of the binding domain. C. Engineered botulinum molecule with double binding domains causes enhanced cleavage of SNAP25 in neuronal cell cultures. Western immunoblot (left panel) and quantification (right panel) show comparative cleavage of SNAP-25 in a neuronal cell culture treated with either the single-binding or double-binding variants of bonded botulinum molecule.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The present invention provides novel neurotoxins and compositions comprising the same. The neurotoxins are useful in therapy, particularly for preventing, regulating or reducing neuropathic pain or sweating. Methods and kits for producing the neurotoxins are also provided.

### Neurotoxins

Neurotoxins comprising a SNARE peptidase domain, a translocation domain and a neuronal binding domain are provided herein, wherein two of the domains are present within a disulphide-linked polypeptide that is covalently linked to the third domain via an isopeptide bond.

The term “neurotoxin” is used herein to refer to a protein complex that comprises at least the following three functional domains: a SNARE peptidase domain, a translocation domain and a neuronal binding domain. Neurotoxins described herein utilize these domains to specifically target neural components and inhibit neuronal communication across a synapse. In other words, the neurotoxins act as neuronal blockers or neuronal modulators. The neurotoxins are not identical to neurotoxins found in nature due to the presence of an isopeptide bond between two of the domains. A neurotoxin described herein may therefore also be referred to as a “modified neurotoxin”, a “neuronal blocker” and/or a “neuronal modulator”.

The terms “neurotoxin” and “clostridial neurotoxin” are used interchangeably herein. The neurotoxins described herein are typically made up of botulinum neurotoxin domains. A neurotoxin described herein may therefore be referred to as a “botulinum neurotoxin”, or a “modified botulinum neurotoxin”. These terms are used interchangeably and encompass neurotoxins that have the same SNARE peptidase domain, translocation domain and neuronal binding domain combinations as natural botulinum neurotoxins (e.g. have Bot/A SNARE peptidase, translocation and neuronal binding domains), as well neurotoxins that have a combination of domains that is not found in nature. The latter neurotoxins may also be referred to as “Botulinum neurotoxin chimeras” and are also referred to as “hybrid” or “chimeric” herein. Several Botulinum neurotoxin chimeras have been described, see for example, WO2018/132423 and WO2018/060351).

As would be clear to a person of skill in the art, chimeric botulinum neurotoxins include neurotoxins wherein each domain (e.g. SNARE peptidase domain, translocation domain, neuronal binding domain etc) is a domain that is naturally occurring (e.g. one of the sequences provided herein), but the combination of domains is not found in nature (e.g. the chimeric Bot has the SNARE peptidase domain and translocation domain of Botulinum neurotoxin type A, and the neuronal binding domain of botulinum neurotoxin type C). In addition, chimeric botulinum neurotoxins also include neurotoxins wherein at least one of the domains is itself chimeric (e.g. the neuronal binding domain is a hybrid domain that is a combination of sequences from a botulinum neurotoxin type C neuronal binding domain and sequences from a botulinum neurotoxin type D neuronal binding domain, for example).

As used herein, the term “botulinum neurotoxin” and its abbreviation “Bot” are used interchangeably. For example, botulinum neurotoxin type A may also be referred to herein as “botulinum neurotoxin A” or “Bot/A”.

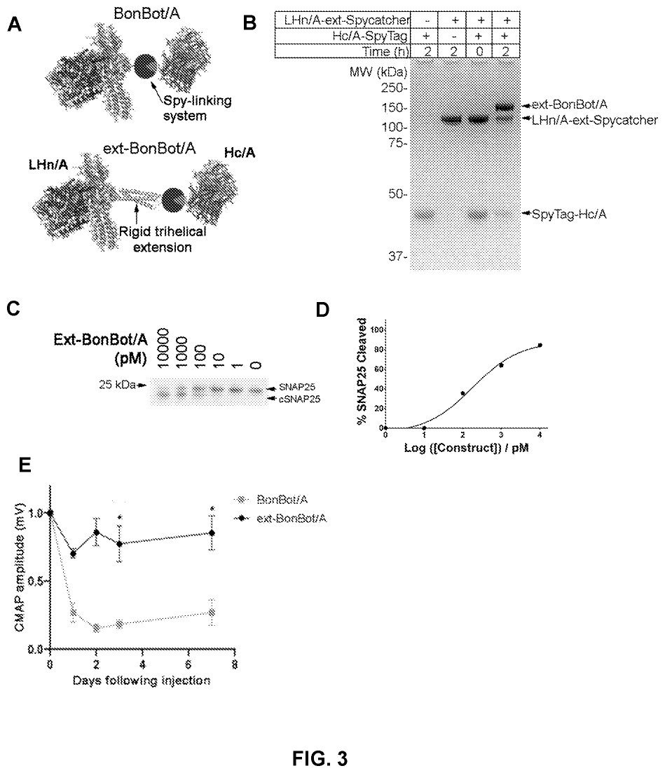

The novel neurotoxins provided herein are made up of functional botulinum domains and are referred to as “Bonded Botulinum” or “BonBot” (e.g. BonBot/A). For the avoidance of doubt, BonBot/A is made up of a botulinum neurotoxin type A SNARE peptidase domain, a botulinum neurotoxin type A translocation domain and a botulinum neurotoxin type A neuronal binding domain wherein two of the domains are present within a disulphide-linked polypeptide that is covalently linked to the third domain via an isopeptide bond (FIG. 3A).

The novel neurotoxins may also include additional domains such as an extra neuronal binding domain (such that e.g. the neurotoxin comprises a botulinum neurotoxin type A SNARE peptidase domain, a botulinum neurotoxin type A translocation domain and two botulinum neurotoxin type A neuronal binding domains wherein two of the domains (typically the botulinum neurotoxin type A SNARE peptidase domain, a botulinum neurotoxin type A translocation domain) are present within a disulphide-linked polypeptide that is covalently linked to the third domain (typically the first botulinum neurotoxin type A neuronal binding domain) via an isopeptide bond, and optionally wherein the disulphide-linked polypeptide also is covalently linked to the fourth domain (typically the second botulinum neurotoxin type A neuronal binding domain) via a second isopeptide bond. See for example BonBot/AA described in the examples section below.

The term polypeptide “domain” refers to a portion of a polypeptide sequence that can evolve, function and exist independently of the rest of the polypeptide chain. Typically, each domain within a polypeptide may form a compact three-dimensional structure and often can be independently stable and folded.

As used herein, the term “neuronal binding domain” (abbreviated to “Nbd”) refers to a protein domain within a polypeptide, wherein the protein domain facilitates binding of the polypeptide to neuronal cells. Typically, the neuronal binding domain interacts with a receptor on the surface of the target neuronal cell. In this context, the neuronal binding domain may also be considered as a ligand for the corresponding receptor. Accordingly, the terms “neuronal binding domain” and “ligand” are used herein as alternative terminology to describe a neuronal binding domain (unless the context specifies otherwise).

In the context of neurotoxins, the phrase “neuronal binding domain” encompasses botulinum neurotoxin binding domains also known as Hc domains. The neuronal binding domain of the neurotoxin provided herein may therefore be:

(i) a botulinum neurotoxin binding domain (Bot/Nbd) type A;

(ii) a Bot/Nbd type B;

(iii) a Bot/Nbd type C;

(iv) a Bot/Nbd type D;

(v) a Bot/Nbd type E;

(vi) a Bot/Nbd type F;

(vii) a Bot/Nbd type G;

(viii) a Bot/Nbd type X;

(ix) chimeric Bots Nbds (e.g. CD, DC, etc.).

In the context of binding domains, the term “activity” is used to refer to the physiological function of the binding domain i.e. its binding capacity for its target (e.g. receptor).

“Botulinum neurotoxin binding domain (Bot/Nbd) type A” refers to a polypeptide that retains the functional binding capacity of a botulinum type A binding domain i.e. it is capable of binding neuronal ganglioside GT1b and synaptic vesicle protein SV2. The neuronal-binding domain derived from botulinum neurotoxin type A (EC:3.4.24.69) is coded by amino acids 874-1296 from the Protein Identifier P10845-1. A person of skill in the art is readily aware of how to identify polypeptides having Bot/Nbd type A activity using routine experiments known in the art. A suitable experiment for identifying functional Bot/Nbd type A polypeptides is summarised in [2].

In one embodiment, the Bot/Nbd type A comprises the amino acid sequence shown in SEQ ID NO: 6, or functional variants (or functional fragments) thereof. Such variants may be naturally occurring (e.g. allelic), synthetic, or synthetically improved functional variants of SEQ ID NO:6. The term “variant” also encompasses homologues.

Functional variants will typically contain only conservative substitutions of one or more amino acids of SEQ ID NO:6, or substitution, deletion or insertion of non-critical amino acids in non-critical regions of the protein. A functional variant of SEQ ID NO:6 may therefore be a conservative amino acid sequence variant of SEQ ID NO:6, wherein the variant has Bot/Nbd type A activity.

Non-functional variants are amino acid sequence variants of SEQ ID NO: 6 that do not have Bot/Nbd type A activity. Non-functional variants will typically contain a non-conservative substitution, a deletion, or insertion or premature truncation of the amino acid sequence of SEQ ID NO:6 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in Bot/Nbd type A is provided in [2]. Accordingly, a person of skill in the art would readily be able to identify amino acids that may be substituted to provide functional variants (or functional fragments), such as conservative amino acid sequence variants, of SEQ ID NO:6.

A polypeptide having Bot/Nbd type A activity may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:6, or portions or fragments thereof. Suitably, percent identity can be calculated as the percentage of identity to the entire length of the reference sequence (e.g. SEQ ID NO:6), or portions or fragments thereof.

“Botulinum neurotoxin binding domain (Bot/Nbd) type B” refers to a polypeptide that retains the functional binding capacity of a botulinum type B binding domain i.e. it is capable of binding neuronal gangliosides and synaptic vesicle protein synaptotagmin. A person of skill in the art is readily aware of how to identify polypeptides having Bot/Nbd type B activity using routine experiments known in the art. A suitable experiment for identifying functional Bot/Nbd type B polypeptides is summarised in [2].

In one embodiment, the polypeptide having Bot/Nbd type B activity comprises the amino acid sequence shown in SEQ ID NO: 8, or functional variants (or functional fragments) thereof. Such variants may be naturally occurring (e.g. allelic), synthetic, or synthetically improved functional variants of SEQ ID NO:8. The term “variant” also encompasses homologues.

Functional variants will typically contain only conservative substitutions of one or more amino acids of SEQ ID NO:8, or substitution, deletion or insertion of non-critical amino acids in non-critical regions of the protein. A functional variant of SEQ ID NO:8 may therefore be a conservative amino acid sequence variant of SEQ ID NO:8, wherein the variant has Bot/Nbd type B activity.

Non-functional variants are amino acid sequence variants of SEQ ID NO: 8 that do not have Bot/Nbd type B activity. Non-functional variants will typically contain a non-conservative substitution, a deletion, or insertion or premature truncation of the amino acid sequence of SEQ ID NO:8 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in Bot/Nbd type B is provided in [2]. Accordingly, a person of skill in the art would readily be able to identify amino acids that may be substituted to provide functional variants (or functional fragments), such as conservative amino acid sequence variants, of SEQ ID NO:8.

A polypeptide having Bot/Nbd type B activity may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:8, or portions or fragments thereof. Suitably, percent identity can be calculated as the percentage of identity to the entire length of the reference sequence (e.g. SEQ ID NO:8), or portions or fragments thereof.

“Botulinum neurotoxin binding domain (Bot/Nbd) type C” refers to a polypeptide that retains the functional binding capacity of a botulinum type C binding domain i.e. it is capable of binding neuronal gangliosides. A person of skill in the art is readily aware of how to identify polypeptides having Bot/Nbd type C activity using routine experiments known in the art. A suitable experiment for identifying functional Bot/Nbd type C polypeptides is summarised in [2].

In one embodiment, the polypeptide having Bot/Nbd type C activity comprises the amino acid sequence shown in SEQ ID NO: 7, or functional variants (or functional fragments) thereof. Such variants may be naturally occurring (e.g. allelic), synthetic, or synthetically improved functional variants of SEQ ID NO:7. The term “variant” also encompasses homologues.

Functional variants will typically contain only conservative substitutions of one or more amino acids of SEQ ID NO:7, or substitution, deletion or insertion of non-critical amino acids in non-critical regions of the protein. A functional variant of SEQ ID NO:7 may therefore be a conservative amino acid sequence variant of SEQ ID NO:7, wherein the variant has Bot/Nbd type C activity.

Non-functional variants are amino acid sequence variants of SEQ ID NO: 7 that do not have Bot/Nbd type C activity. Non-functional variants will typically contain a non-conservative substitution, a deletion, or insertion or premature truncation of the amino acid sequence of SEQ ID NO:7 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in Bot/Nbd type C is provided in [2]. Accordingly, a person of skill in the art would readily be able to identify amino acids that may be substituted to provide functional variants (or functional fragments), such as conservative amino acid sequence variants, of SEQ ID NO:7.

A polypeptide having Bot/Nbd type C activity may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:7, or portions or fragments thereof. Suitably, percent identity can be calculated as the percentage of identity to the entire length of the reference sequence (e.g. SEQ ID NO:7), or portions or fragments thereof.

“Botulinum neurotoxin binding domain (Bot/Nbd) type D” refers to a polypeptide that retains the functional binding capacity of a botulinum type D binding domain i.e. it is capable of binding neuronal gangliosides. A person of skill in the art is readily aware of how to identify polypeptides having Bot/Nbd type D activity using routine experiments known in the art. A suitable experiment for identifying functional Bot/Nbd type D polypeptides is summarised in [2].

In one embodiment, the polypeptide having Bot/Nbd type D activity comprises the amino acid sequence shown in SEQ ID NO: 9, or functional variants (or functional fragments) thereof. Such variants may be naturally occurring (e.g. allelic), synthetic, or synthetically improved functional variants of SEQ ID NO:9. The term “variant” also encompasses homologues.

Functional variants will typically contain only conservative substitutions of one or more amino acids of SEQ ID NO:9, or substitution, deletion or insertion of non-critical amino acids in non-critical regions of the protein. A functional variant of SEQ ID NO:9 may therefore be a conservative amino acid sequence variant of SEQ ID NO:9, wherein the variant has Bot/Nbd type D activity.

Non-functional variants are amino acid sequence variants of SEQ ID NO: 9 that do not have Bot/Nbd type D activity. Non-functional variants will typically contain a non-conservative substitution, a deletion, or insertion or premature truncation of the amino acid sequence of SEQ ID NO:9 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in Bot/Nbd type D is provided in [2]. Accordingly, a person of skill in the art would readily be able to identify amino acids that may be substituted to provide functional variants (or functional fragments), such as conservative amino acid sequence variants, of SEQ ID NO:9.

A polypeptide having Bot/Nbd type D activity may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:9, or portions or fragments thereof. Suitably, percent identity can be calculated as the percentage of identity to the entire length of the reference sequence (e.g. SEQ ID NO:9), or portions or fragments thereof.

“Botulinum neurotoxin binding domain (Bot/Nbd) type E” refers to a polypeptide that retains the functional binding capacity of a botulinum type E binding domain i.e. it is capable of binding synaptic vesicle protein SV2 and neuronal gangliosides. A person of skill in the art is readily aware of how to identify polypeptides having Bot/Nbd type E activity using routine experiments known in the art. A suitable experiment for identifying functional Bot/Nbd type E polypeptides is summarised in [2].

In one embodiment, the polypeptide having Bot/Nbd type E activity comprises the amino acid sequence shown in SEQ ID NO: 10, or functional variants (or functional fragments) thereof. Such variants may be naturally occurring (e.g. allelic), synthetic, or synthetically improved functional variants of SEQ ID NO:10. The term “variant” also encompasses homologues.

Functional variants will typically contain only conservative substitutions of one or more amino acids of SEQ ID NO:10, or substitution, deletion or insertion of non-critical amino acids in non-critical regions of the protein. A functional variant of SEQ ID NO:10 may therefore be a conservative amino acid sequence variant of SEQ ID NO:10, wherein the variant has Bot/Nbd type E activity.

Non-functional variants are amino acid sequence variants of SEQ ID NO: 10 that do not have Bot/Nbd type E activity. Non-functional variants will typically contain a non-conservative substitution, a deletion, or insertion or premature truncation of the amino acid sequence of SEQ ID NO: 10 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in Bot/Nbd type E is provided in [2]. Accordingly, a person of skill in the art would readily be able to identify amino acids that may be substituted to provide functional variants (or functional fragments), such as conservative amino acid sequence variants, of SEQ ID NO:10.

A polypeptide having Bot/Nbd type E activity may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:10, or portions or fragments thereof. Suitably, percent identity can be calculated as the percentage of identity to the entire length of the reference sequence (e.g. SEQ ID NO:10), or portions or fragments thereof.

“Botulinum neurotoxin binding domain (Bot/Nbd) type F” refers to a polypeptide that retains the functional binding capacity of a botulinum type F binding domain i.e. it is capable of binding synaptic vesicle protein SV2 and neuronal gangliosides. A person of skill in the art is readily aware of how to identify polypeptides having Bot/Nbd type F activity using routine experiments known in the art. A suitable experiment for identifying functional Bot/Nbd type F polypeptides is summarised in [2].

In one embodiment, the polypeptide having Bot/Nbd type F activity comprises the amino acid sequence shown in SEQ ID NO: 11, or functional variants (or functional fragments) thereof. Such variants may be naturally occurring (e.g. allelic), synthetic, or synthetically improved functional variants of SEQ ID NO:11. The term “variant” also encompasses homologues.

Functional variants will typically contain only conservative substitutions of one or more amino acids of SEQ ID NO:11, or substitution, deletion or insertion of non-critical amino acids in non-critical regions of the protein. A functional variant of SEQ ID NO:11 may therefore be a conservative amino acid sequence variant of SEQ ID NO:11, wherein the variant has Bot/Nbd type F activity.

Non-functional variants are amino acid sequence variants of SEQ ID NO: 11 that do not have Bot/Nbd type F activity. Non-functional variants will typically contain a non-conservative substitution, a deletion, or insertion or premature truncation of the amino acid sequence of SEQ ID NO:11 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in Bot/Nbd type F is provided in [2]. Accordingly, a person of skill in the art would readily be able to identify amino acids that may be substituted to provide functional variants (or functional fragments), such as conservative amino acid sequence variants, of SEQ ID NO:11.

A polypeptide having Bot/Nbd type F activity may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:11, or portions or fragments thereof. Suitably, percent identity can be calculated as the percentage of identity to the entire length of the reference sequence (e.g. SEQ ID NO:11), or portions or fragments thereof.

“Botulinum neurotoxin binding domain (Bot/Nbd) type G” refers to a polypeptide that retains the functional binding capacity of a botulinum type G binding domain i.e. it is capable of binding neuronal gangliosides and synaptotagmin. A person of skill in the art is readily aware of how to identify polypeptides having Bot/Nbd type G activity using routine experiments known in the art. A suitable experiment for identifying functional Bot/Nbd type G polypeptides is summarised in [2].

In one embodiment, the polypeptide having Bot/Nbd type G activity comprises the amino acid sequence shown in SEQ ID NO: 12, or functional variants (or functional fragments) thereof. Such variants may be naturally occurring (e.g. allelic), synthetic, or synthetically improved functional variants of SEQ ID NO:12. The term “variant” also encompasses homologues.

Functional variants will typically contain only conservative substitutions of one or more amino acids of SEQ ID NO:12, or substitution, deletion or insertion of non-critical amino acids in non-critical regions of the protein. A functional variant of SEQ ID NO:12 may therefore be a conservative amino acid sequence variant of SEQ ID NO:12, wherein the variant has Bot/Nbd type G activity.

Non-functional variants are amino acid sequence variants of SEQ ID NO:12 that do not have Bot/Nbd type G activity. Non-functional variants will typically contain a non-conservative substitution, a deletion, or insertion or premature truncation of the amino acid sequence of SEQ ID NO:12 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in Bot/Nbd type G is provided in [2]. Accordingly, a person of skill in the art would readily be able to identify amino acids that may be substituted to provide functional variants (or functional fragments), such as conservative amino acid sequence variants, of SEQ ID NO:12.

A polypeptide having Bot/Nbd type G activity may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:12, or portions or fragments thereof. Suitably, percent identity can be calculated as the percentage of identity to the entire length of the reference sequence (e.g. SEQ ID NO:12), or portions or fragments thereof.

In some examples, the neurotoxin provided herein may comprise more than one neuronal binding domain, e.g. two neuronal binding domains. The plurality of neuronal binding domain may be the same (e.g. both be botulinum neurotoxin type A neuronal binding domains, see BonBot/AA described herein) or they may be different neuronal binding domains.

The neurotoxins provided herein also comprise a SNARE peptidase domain also known as Light chain (L). As used herein, a “SNARE peptidase domain” refers to protein domain within a polypeptide, wherein the protein domain hydrolyses one more SNAREs. In other words, the protein domain functions as a protease enzyme that performs proteolysis on its substrate, wherein the substrate is a SNARE. The term “enzymatic domain” is used herein as alternative terminology to describe a (SNARE) peptidase domain. Once a neurotoxin is present within a target cell, the SNARE peptidase domain can function as the “toxin” component, as hydrolysis of its SNARE substrate in the cell can result in a blockade in neurotransmitter release.

As used herein, a “botulinum SNARE peptidase domain” refers to a SNARE peptidase domain as found in naturally occurring botulinum neurotoxins (e.g. any one of botulinum neurotoxin types A, B, C, D, E, F, G, X or chimeric botulinum neurotoxins), and functional derivatives (or variants) thereof. It includes functional allelic variants, fragments or portions thereof. For the avoidance of doubt, a SNARE peptidase domain from any one of botulinum neurotoxin types A, B, C, D, E, F, G, X or chimeric botulinum neurotoxins could be present in the neurotoxins presented herein, and could function as the “toxin” component of the neurotoxin once in the target cell.

In its broadest sense, a “botulinum SNARE peptidase domain” therefore refers to a polypeptide that retains the functional peptidase activity of a botulinum SNARE peptidase domain i.e. it is capable of catalysing the proteolysis of at least one of three SNAREs, namely VAMP/synaptobrevin, SNAP25 or syntaxin. A person of skill in the art is readily aware of how to identify a botulinum SNARE peptidase domain polypeptide using routine experiments known in the art. A suitable experiment for identifying functional botulinum SNARE peptidase domains is summarised in [14].

In one embodiment, a botulinum SNARE peptidase domain comprises the amino acid sequence shown in SEQ ID NO: 3, or functional variants (or functional fragments) thereof.

Such variants may be naturally occurring (e.g. allelic), synthetic, or synthetically improved functional variants of SEQ ID NO:3. The term “variant” also encompasses homologues.

In one embodiment, the term “botulinum SNARE peptidase domain” includes isozymes and allozymes of a polypeptide comprising the amino acid sequence shown in SEQ ID NO: 3.

Functional variants will typically contain only conservative substitutions of one or more amino acids of SEQ ID NO:3, or substitution, deletion or insertion of non-critical amino acids in non-critical regions of the protein. A functional variant of SEQ ID NO:3 may therefore be a conservative amino acid sequence variant of SEQ ID NO:3, wherein the variant has botulinum SNARE peptidase activity.

Non-functional variants are amino acid sequence variants of SEQ ID NO: 3 that do not have botulinum SNARE peptidase activity. Non-functional variants will typically contain a non-conservative substitution, a deletion, or insertion or premature truncation of the amino acid sequence of SEQ ID NO:3 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in a botulinum SNARE peptidase domain is provided in [2]. Accordingly, a person of skill in the art would readily be able to identify amino acids that may be substituted to provide functional variants (or functional fragments), such as conservative amino acid sequence variants, of SEQ ID NO:3.

A SNARE peptidase domain according to the invention may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:3, or portions or fragments thereof. Suitably, percent identity can be calculated as the percentage of identity to the entire length of the reference sequence (e.g. SEQ ID NO:3), or portions or fragments thereof.

The amino acid sequence shown in SEQ ID NO:3 is that of a Botulinum neurotoxin type A SNARE peptidase domain. However, other Botulinum neurotoxin SNARE peptidase domains may also be used, e.g. that of botulinum neurotoxin type B, C, D, E, F, G, X or chimeric botulinum neurotoxins. Variants of the SNARE peptidase domain of botulinum type B, C, D, E, F, G, X are equally covered, as for the SNARE peptidase domain of botulinum type A (SEQ ID NO:3), and therefore the paragraphs above apply equally for SEQ ID NO:3 and the SNARE peptidase domain of botulinum type B, C, D, E, F, G, X or chimeric botulinum neurotoxins.

The neurotoxins provided herein also comprise a translocation domain. As used herein, a “translocation domain” refers to protein domain within a polypeptide, wherein the protein domain facilitates translocation of the neurotoxin into the cytosol of the target cell. In the context of the invention, and in practical terms, the translocation domain is typically derived from the same origin as the SNARE peptidase domain (as the SNARE peptidase domain and translocation domain of each neurotoxin are typically inseparable in terms of structure and function). By way of example, a botulinum type A SNARE peptidase domain is typically used with a botulinum type A translocation domain; a botulinum type B SNARE peptidase domain is typically used with a botulinum type B translocation domain; a botulinum type C SNARE peptidase domain is typically used with a botulinum type C translocation domain; a botulinum type D SNARE peptidase domain is typically used with a botulinum type D translocation domain; a botulinum type E SNARE peptidase domain is typically used with a botulinum type E translocation domain; a botulinum type F SNARE peptidase domain is typically used with a botulinum type F translocation domain; botulinum type G SNARE peptidase domain is typically used with a botulinum type G translocation domain; and a tetanus SNARE peptidase domain is typically used with a tetanus translocation domain.

The translocation domain of the invention may be a botulinum neurotoxin translocation domain. As used herein, a “botulinum translocation domain” refers to a translocation domain as found in naturally occurring botulinum neurotoxins (e.g. any one of botulinum neurotoxin types A, B, C, D, E, F, G and X or chimeric botulinum neurotoxins), and functional derivatives (or variants) thereof. It includes functional allelic variants, fragments or portions thereof.

In its broadest sense, a “botulinum translocation domain” therefore refers to a polypeptide that retains the functional translocation activity of a botulinum translocation domain i.e. it is capable of facilitating the entry of the SNARE peptidase into the cytosol of the target cell. A person of skill in the art is readily aware of how to identify botulinum translocation domain polypeptides using routine experiments known in the art. A suitable experiment for identifying functional botulinum translocation domain polypeptides is summarised in [18].

In one embodiment, the translocation domain polypeptide comprises the amino acid sequence shown in SEQ ID NO:4, or functional variants (or functional fragments) thereof. Such variants may be naturally occurring (e.g. allelic), synthetic, or synthetically improved functional variants of SEQ ID NO:4. The term “variant” also encompasses homologues.

Functional variants will typically contain only conservative substitutions of one or more amino acids of SEQ ID NO:4, or substitution, deletion or insertion of non-critical amino acids in non-critical regions of the protein. A functional variant of SEQ ID NO:4 may therefore be a conservative amino acid sequence variant of SEQ ID NO:4, wherein the variant is capable of facilitating endocytosis of the neurotoxin into the cytosol of a target cell.

Non-functional variants are amino acid sequence variants of SEQ ID NO: 4 that are not capable of facilitating endocytosis of the neurotoxin into the cytosol of the target cell. Non-functional variants will typically contain a non-conservative substitution, a deletion, or insertion or premature truncation of the amino acid sequence of SEQ ID NO:4 or a substitution, insertion or deletion in critical amino acids or critical regions. Methods for identifying functional and non-functional variants (e.g. functional and non-functional allelic variants) are well known to a person of ordinary skill in the art.

A summary of the critical and non-critical amino acids in translocation domains is provided in [3]. Accordingly, a person of skill in the art would readily be able to identify amino acids that may be substituted to provide functional variants (or functional fragments), such as conservative amino acid sequence variants, of SEQ ID NO:4.

A translocation domain according to the invention may comprise an amino acid sequence having at least about 60%, 65%, 70%, 75%, 80%, 85%, 90%, 91%, 92%, 93%, 94%, 95%, 96%, 97%, 98%, 99% or 100% identity to the amino acid sequence of SEQ ID NO:4, or portions or fragments thereof. Suitably, percent identity can be calculated as the percentage of identity to the entire length of the reference sequence (e.g. SEQ ID NO:4), or portions or fragments thereof.

In one example, the translocation domain is part of a longer sequence such as that shown in SEQ ID NO:5. This is a variant sequence of SEQ ID NO:4 which still retains functional activity (i.e. it is still capable of facilitating endocytosis of the neurotoxin into the cytosol of the target cell). It is therefore a bonafide botulinum neurotoxin type A translocation domain variant, and is encompassed by the claims. It is noted that the sequence of SEQ ID NO:5 includes an extension amino acid (SEQ ID NO:18) which is not naturally found in neurotoxins. As will be described in more detail below, neurotoxins of the invention that comprise additional extension sequence are very effective at reducing neuropathic pain. Accordingly, in the context of the invention, translocation domains having at least 80% sequence identity to SEQ ID NO:4 or SEQ ID NO:5 may be used. As described elsewhere, other functional variants may also be used.

Preferably, the SNARE peptidase domain and the translocation domain are from the same botulinum neurotoxin. In one example, the SNARE peptidase domain and the translocation domain are therefore both botulinum type A domains. In other words, the SNARE peptidase domain is a botulinum type A SNARE peptidase domain and the translocation domain is a botulinum type A translocation domain. The SNARE peptidase with corresponding translocation domain comprise the sequence of botulinum neurotoxin type A (EC:3.4.24.69) amino acids 1-852 from the Protein Identifier P10845-1. In this example, the SNARE peptidase domain may have at least 80% (e.g. at least 90, at least 95% etc) sequence identity to the amino acid sequence of SEQ ID NO: 3, and the translocation domain may have at least 80% (e.g. at least 90, at least 95%) sequence identity to the amino acid sequence of SEQ ID NO: 4 or SEQ ID NO:5. For example, the SNARE peptidase domain may comprise the amino acid sequence of SEQ ID NO:3 and the translocation domain may comprise the amino acid sequence of SEQ ID NO: 4. Alternatively, the SNARE peptidase domain may comprise the amino acid sequence of SEQ ID NO:3 and the (extended) translocation domain may comprise the amino acid sequence of SEQ ID NO: 5.

The neurotoxins described herein comprise a SNARE peptidase domain, a translocation domain and a neuronal binding domain. As stated elsewhere herein, two of these domains are present within a disulphide-linked polypeptide. In other words, the neurotoxins described herein comprise a polypeptide having two domains that are linked to each other by a disulphide bond. One of the domains is thus N-terminal of the disulphide bond, whilst the other domain is C-terminal of the disulphide bond. In the novel neurotoxins described herein, the polypeptide having two domains that are linked to each other by a disulphide bond is covalently linked to the third domain via an isopeptide bond.

Preferably, the SNARE peptidase domain and the translocation domain are present within a disulphide-linked polypeptide. In other words, in a preferred embodiment, these are the two domains that are linked to each other by a disulphide bond, thereby forming a single polypeptide sequence. This is the preferred arrangement as the SNARE peptidase domain and translocation domain of each neurotoxin are typically inseparable in terms of structure and function (and therefore are typically localised in close proximity with each other via a disulphide bond).

In one example, the SNARE peptidase domain and the translocation domain are from the same botulinum neurotoxin and are joined via a disulphide bond as in a naturally occurring botulinum neurotoxin. If the SNARE peptidase domain and the translocation domain are joined by a peptide bond between the amino acid chains, preferably, there is a nicking site in the amino acid sequence between the SNARE peptidase domain and the translocation domain which is recognised by a protease to cause cleavage of the amino acid sequence between the two parts. In one embodiment, the nicking site is a thrombin site which can be cleaved by thrombin.

Typically, when the SNARE peptidase domain and the translocation domain are present within a disulphide-linked polypeptide, the SNARE peptidase domain is located N-terminal (in relative terms) to the translocation domain. In other words, going from N-terminal to C-terminal, the order of components in the disulphide-linked polypeptide is typically: SNARE peptidase domain, disulphide bond, translocation domain, optional extension sequence.

The N-terminus of a protein (also known as the amino-terminus, NH₂-terminus, N-terminal end or amine-terminus) is the start of a protein or polypeptide terminated by an amino acid with a free amine group (—NH₂). By convention, peptide sequences are written N-terminus to C-terminus (from left to right). The C-terminus (also known as the carboxyl-terminus, carboxy-terminus, C-terminal tail, C-terminal end, or COOH-terminus) is the end of an amino acid chain (protein or polypeptide), terminated by a free carboxyl group (—COOH).

As used herein, the terms “N-terminal” and “C-terminal” are used to describe the relative position of e.g. a domain within a polypeptide. Accordingly, a domain that is “N-terminal” is positioned closer (in relative terms) to the N-terminus than to the C-terminus of the polypeptide. Conversely, a domain that is “C-terminal” is positioned (in relative terms) closer to the C-terminus than to the N-terminus of the polypeptide. As used herein, the term “positioned” refers to the location of the e.g. domain within the linear amino acid sequence of the polypeptide.

The terms “N-terminal” and “C-terminal” can be used to describe the relative position of two or more domains within a polypeptide. In this context, a domain that is “N-terminal” is positioned closer (in relative terms) to the N-terminus of the polypeptide than a domain that is “C-terminal”. Conversely, a domain that is “C-terminal” is positioned closer (in relative terms) to the C-terminus of the polypeptide than a domain that is “N-terminal”.

A domain that is “N-terminal” may be, but does not have to be, at the N-terminus of the polypeptide (i.e. it may be, but does not have to be, at the start of the polypeptide terminated by an amino acid with a free amine group). In other words, the first amino acid of an N-terminal domain does not need to be (but may be) the first amino acid of the polypeptide. This means that there may be other amino acids, polypeptide domains (e.g. tags such as HA tags) etc between the N-terminus of the polypeptide and the start of the “N-terminal” domain (provided that the domain is positioned closer to the N-terminus than to the C-terminus of the polypeptide; or when used to describe the relative positions of two or more domains, provided that the domain is positioned closer to the N-terminus than a domain that is “C-terminal”).

Likewise, a domain that is “C-terminal” may be, but does not have to be, at the C-terminus of the polypeptide (i.e. it may be, but does not have to be, at the end of the polypeptide terminated by any amino acid with a free carboxyl group). In other words, the last amino acid of a C-terminal domain does not need to be (but may be) the last amino acid of the polypeptide. This means that there may be other amino acids, polypeptide domains etc (e.g. tags) between the C-terminus of the polypeptide and the end of the “C-terminal” domain (provided that the domain is positioned closer to the C-terminus than to the N-terminus of the polypeptide; or when used to describe the relative positions of two or more domains, provided that the domain is positioned closer to the C-terminus than a domain that is “N-terminal”).

Polypeptides comprising an N-terminal polypeptide domain (A) and a C-terminal polypeptide domain (B) are conventionally written as A-B i.e. N-terminal to C-terminal (left to right). By way of example, a polypeptide comprising an N-terminal SNARE peptidase domain (and a C-terminal translocation domain will conventionally be written as LHn.

In the context of the neurotoxins described herein, the disulphide-linked polypeptide (comprising two of the neurotoxin domains as described above) is covalently linked to the third domain via an isopeptide bond. An isopeptide bond is an amide bond that can form for example between the carboxyl group of one amino acid and the amino group of another. At least one of these joining groups is part of the side chain of one of these amino acids. Lysine for example has an amino group on its side chain and aspartic acid has a carboxy group on its side chain. Appropriate joining groups for isopeptide bond formation are well known in the art and include isopeptide bond formation between two amino acid side chains (wherein, in the context of the invention, one of the amino acids would be part of the disulphide-linked polypeptide having two of the neurotoxin domains, and the other would be part of the polypeptide that comprises the third neurotoxin domain). In one example, the isopeptide bond may form between two amino acid polar side chains. Alternatively, the isopeptide bond may form between an amino acid basic side chain and a polar side chains: such as an aspartate, glutamate, asparagine or glutamine side chain. For example, the isopeptide bond may form between lysine and aspartate side chains.

Isopeptide bond formation may occur between the C-terminus of the disulphide-linked polypeptide (e.g. LHn) and the corresponding N-terminus of the polypeptide comprising the third domain (e.g. Hc) (or vice versa, depending on which terminal amino acids that are capable of isopeptide bond formation). In other words, at least one of the N-terminus or the C-terminus of the disulphide-linked polypeptide must be capable of isopeptide bond formation with the cognate terminus of the polypeptide comprising the third domain, such that isopeptide bond formation results in the disulphide-linked polypeptide being covalently linked to the third domain.

In one non-limiting example, the isopeptide bond is generated using a bipartite bonding system, such as the Spycatcher-Spytag system described in the examples section below. In another non-limiting example, the isopeptide bond is generated using an alternative bipartite bonding system such as Snoopcatcher-Snooptag, Sdycatcher-Sdytag, Pilin-C-isopeptag-C, or Pilin-N-isopeptag-N. These systems are advantageous as they provide spatial distancing between the disulphide-linked polypeptide and the third domain of the neurotoxin. As shown herein, this spatial distancing may be further increased using optional extension sequences such as spacer peptides. Spatial distancing between the disulphide-linked polypeptide (e.g. SNARE peptidase and translocation domain) and the third domain (e.g. neuronal binding domain) of the neurotoxin is shown to be beneficial as it reduces the paralytic properties of the neurotoxin whilst retaining pain relief properties of the neurotoxin.

Accordingly, in one example, the isopeptide bond is formed between a peptide and its cognate protein, wherein the peptide is attached to the disulphide-linked polypeptide and the cognate protein is attached to the third domain; or the cognate protein attached to the disulphide-linked polypeptide (LHn) and the peptide is attached to the third domain (Hc).

In this context, the peptide may be attached to the C-terminus of the disulphide-linked polypeptide, and the cognate protein may be attached to the N-terminus of the polypeptide comprising the third domain. In this example, the peptide may be attached to the C-terminal domain in the disulphide-linked polypeptide (e.g. the translocation domain), or it may be attached to a spacer peptide that is between the C-terminus of the disulphide-linked polypeptide and the C-terminal domain. Similarly, in this example, the cognate protein may be attached to the N-terminus of the third domain, or it may be attached to a spacer peptide that is between the N-terminus of the polypeptide comprising the third domain and the third domain itself.

In an alternative example, the cognate protein may be attached to the C-terminus of the disulphide-linked polypeptide, and the peptide may be attached to the N-terminus of the polypeptide comprising the third domain. In this example, the cognate protein may be attached to the C-terminal domain in the disulphide-linked polypeptide (e.g. the translocation domain), or it may be attached to a spacer peptide that is between the C-terminus of the disulphide-linked polypeptide and the C-terminal domain. Similarly, in this example, the peptide may be attached to the N-terminus of the third domain, or it may be attached to a spacer peptide that is between the N-terminus of the polypeptide comprising the third domain and the third domain itself.

As stated above, the peptide may be attached to the disulphide-linked polypeptide and the cognate protein may be attached to the third domain; or conversely the cognate protein may be attached to the disulphide-linked polypeptide when the peptide is attached to the third domain. In other words, the peptide and disulphide-linked polypeptide may be one fusion protein, whilst the cognate protein and third domain are a distinct fusion protein; or the cognate protein and disulphide-linked polypeptide may be one fusion protein, whilst the peptide and third domain are a distinct fusion protein.

As used herein, “attached” refers to direct or indirect attachment i.e. in the context of the fusion proteins described above, the peptide may be immediately adjacent to the corresponding neurotoxin domain, or alternatively, there may be one or more amino acids between the peptide and its corresponding neurotoxin domain. For example, one or more amino acids may be needed between the peptide and its corresponding domain in order to retain the domain's structural integrity (and/or function) in the resultant neurotoxin complex. Identifying whether or not one or more amino acids is required or optimal is well within the capabilities of a person of skill in the art. Suitable amino acid sequences for this purpose are well known to those skilled in the art.

Similarly, in the context of the fusion proteins described above, the cognate protein may be immediately adjacent to the corresponding neurotoxin domain, or alternatively, there may be one or more amino acids between the cognate protein and its corresponding neurotoxin domain. For example, one or more amino acids may be needed between the cognate protein and its corresponding domain in order to retain the domain's structural integrity (and/or function) in the resultant neurotoxin complex. Identifying whether or not one or more amino acids is required or optimal is well within the capabilities of a person of skill in the art. Suitable amino acid sequences for this purpose are well known to those skilled in the art.

The term “attached” is also used to describe the interaction between the two domains within a disulphide-linked polypeptide (e.g. a polypeptide comprising a SNARE peptidase domain and the translocation domain). In this context, the term “attached” also refers to direct or indirect attachment (i.e. one or more amino acids may be present between the recited domains). Identifying whether or not one or more amino acids is required or optimal is well within the capabilities of a person of skill in the art. Suitable amino acid sequences for this purpose are well known to those skilled in the art.

In any of the above examples, the peptide may comprise an amino acid sequence that has at least 80% (e.g. at least 85%, at least 90%, at least 95%, at least 96%, at least 97%, at least 98%, at least 99% etc) sequence identity to the sequence of SEQ ID NO:1 (the sequence of Spytag). In this example, the cognate protein may comprise an amino acid sequence that has at least 80% (e.g. at least 85%, at least 90%, at least 95%, at least 96%, at least 97%, at least 98%, at least 99% etc) sequence identity to the sequence of SEQ ID NO:2 (the sequence of Spycatcher). In a preferred example, the peptide may comprise the sequence of SEQ ID NO:1 and the cognate protein may comprise the sequence of SEQ ID NO: 2.

For example, the neurotoxin may comprise: a) a disulphide-linked polypeptide comprising the SNARE peptidase domain, the translocation domain and a C-terminal cognate protein comprising the amino acid sequence of SEQ ID NO: 2 (e.g. the order in the polypeptide is SNARE peptidase domain, disulphide bond, translocation domain, SEQ ID NO: 2); and b) the neuronal binding domain attached to an N-terminal peptide comprising the amino acid sequence of SEQ ID NO:1, wherein the disulphide-linked polypeptide is covalently linked to the neuronal binding domain via an isopeptide bond between the cognate protein of a) and peptide of b). In this example, the SNARE peptidase domain and the translocation domain may be a Bot/A SNARE peptidase domain and a Bot/A translocation domain (with the neuronal binding domain optionally also being a Bot/A neuronal binding domain or for example, a Bot/C neuronal binding domain).

Alternatively, the neurotoxin may comprise: a) a disulphide-linked polypeptide comprising the SNARE peptidase domain, the translocation domain and a C-terminal peptide comprising the amino acid sequence of SEQ ID NO: 1 (e.g. the order in the polypeptide is SNARE peptidase domain, disulphide bond, translocation domain, SEQ ID NO: 1); and b) the neuronal binding domain attached to an N-terminal cognate protein comprising the amino acid sequence of SEQ ID NO:2, wherein the disulphide-linked polypeptide is covalently linked to the neuronal binding domain via an isopeptide bond between the peptide of a) and the cognate protein of b). In this example, the SNARE peptidase domain and the translocation domain may be a Bot/A SNARE peptidase domain and a Bot/A translocation domain (with the neuronal binding domain optionally also being a Bot/A neuronal binding domain or for example, a Bot/C neuronal binding domain).

Although details are provided above on the Spycatcher-Spytag system specifically, for the avoidance of doubt, it is noted that alternative appropriate bipartite bonding systems may also be used, for example but not limited to, CnaB-like domains [19], snoopCatcher/snooptag [20], Sdycatcher/Sdytag [21], and/or combinations or variations thereof. Spycatcher and Spytag were initially formed by splitting the CnaB region of the FbaB protein from Streptococcus pyogenes into two parts. However, those two initial parts were slow to react with each other, leading the researchers to make small changes to both parts in order to facilitate binding [19]. It would be possible to use the less-optimal, but still functional, CnaB-like domains iterations of the binding pair for formation of neurotoxin molecules. SnoopCatcher/SnoopTag is another isopeptide bonding pair, created by splitting the RrgA protein from Streptococcus pneumonia into two parts [20]. SdyTag/SdyCatcher has been developed from the CnaB region of the FbaB protein from Streptococcus dysgalactiae to create an alternative isopeptide bonding pair [21]. Although the SdyTag is slower than Spytag at forming complexes, and cannot tolerate pH above 7, it could still potentially be used to form neurotoxin molecules. SdyCatcher is also capable of forming a bond with Spytag, so one could consider the possibility of mixing components from different bonding pairs.

Accordingly, appropriate peptide:cognate protein bonding pairs include: SEQ ID NO:1 with SEQ ID NO:2; SEQ ID NO: 16 with SEQ ID NO:15; SEQ ID NO: 22 with SEQ ID NO: 21; SEQ ID NO: 24 with SEQ ID NO:23; SEQ ID NO: 26 with SEQ ID NO:25; SEQ ID NO:28 with SEQ ID NO:27; SEQ ID NO: 30 with SEQ ID NO:29; and appropriate combinations thereof e.g. SEQ ID NO:25 with SEQ ID NO:1. Other appropriate combinations between peptides and cognate proteins may be identified by a person of skill in the art based on whether the combination is able to form the required isopeptide bond.

The invention is described herein using the pairing of SEQ ID NO:1 (peptide) and SEQ ID NO: 2 (cognate protein). However, as would be clear to a person of skill in the art, this pairing can be replaced with any of the above appropriate pairings when describing the invention. Accordingly, and text that describes the peptide and cognate pairing in the context of SEQ ID NO:1 and SEQ ID NO:2 equally applies to each of the pairing listed in the paragraph above. The terms “SEQ ID NO:1” and “SEQ ID NO:2” can therefore be replaced with an appropriate alternative “peptide” and “cognate protein” pair throughout the text.

In addition, the invention is generally described herein using a single pairing of a peptide and a cognate protein. However, as would be clear to a person of skill in the art, the pairing system described herein is modular, and may also be used to link additional neurotoxin component parts to the molecules described herein. For example, the neurotoxin may be generated by two isopeptide bonds forming between two identical peptide:cognate protein pairs, wherein the two identical cognate proteins are attached (e.g. in series, in other words adjacent to each other) to the disulphide-linked polypeptide and a peptide is attached to the third domain. In this example, the two cognate proteins (being attached (e.g. in series, in other words adjacent to each other) to a disulphide-linked polypeptide comprising the SNARE peptidase domain and the translocation domain) will each bond to a peptide attached to a neuronal binding domain, resulting in a neurotoxin with duplication of its neuronal binding domain (see for example BonBot/AA in the examples).

As would be clear to a person of skill in the art, the neurotoxin may alternatively by generated by two isopeptide bonds forming between two identical peptide:cognate protein pairs, wherein the two identical peptides are attached (e.g. in series, in other words adjacent to each other) to the disulphide-linked polypeptide and a cognate protein is attached to the third domain. In this example, the two peptides (being attached (e.g. in series, in other words adjacent to each other) to a disulphide-linked polypeptide comprising the SNARE peptidase domain and the translocation domain) will each bond to a cognate protein attached to a neuronal binding domain, also resulting in a neurotoxin with duplication of its neuronal binding domain.

As would also be clear to a person of skill in the art, the neurotoxin may alternatively be generated by two distinct isopeptide bonds forming between two distinct peptide;cognate protein pairs, wherein the two cognate proteins are attached (e.g. in series, in other words adjacent to each other) to the disulphide-linked polypeptide and a peptide is attached to the third domain, with a further peptide being attached to an additional fourth domain. In this example, the two cognate proteins (being attached (e.g. in series, in other words adjacent to each other) to a disulphide-linked polypeptide comprising the SNARE peptidase domain and the translocation domain) will bond with a peptide attached to a first neuronal binding domain and with a further peptide attached to a second neuronal binding domain, resulting in a neurotoxin with two neuronal binding domains. This arrangement (comprising two distinct peptide;cognate protein pairs) is particularly useful when generating neurotoxins with heterologous neuronal binding domains (in other words, when the two neuronal binding domains in the resultant neurotoxin are not the same).

In a further example, the neurotoxin may alternatively be generated by two distinct isopeptide bonds forming between two distinct peptide;cognate protein pairs, wherein the two peptides are attached (e.g. in series, in other words adjacent to each other) to the disulphide-linked polypeptide and a cognate protein is attached to the third domain, with a further cognate protein being attached to an additional fourth domain. In this example, the two peptides (being attached (e.g. in series, in other words adjacent to each other) to a disulphide-linked polypeptide comprising the SNARE peptidase domain and the translocation domain) will bond with a cognate protein attached to a first neuronal binding domain and with a further cognate protein attached to a second neuronal binding domain, resulting in a neurotoxin with two neuronal binding domains. This arrangement (comprising two distinct peptide;cognate protein pairs) is particularly useful when generating neurotoxins with heterologous neuronal binding domains (in other words, when the two neuronal binding domains in the resultant neurotoxin are not the same).

In these examples the two peptide;cognate protein pairs may therefore be the same (e.g. both be SEQ ID NO:1 (peptide) and SEQ ID NO: 2 (cognate protein)) or they may be different (e.g. one pair is SEQ ID NO:1 (peptide) and SEQ ID NO: 2 (cognate protein); whilst the other pair is e.g. SEQ ID NO:28 and SEQ ID NO:27). Suitable combinations are readily identifiable to a person of skill in the art.

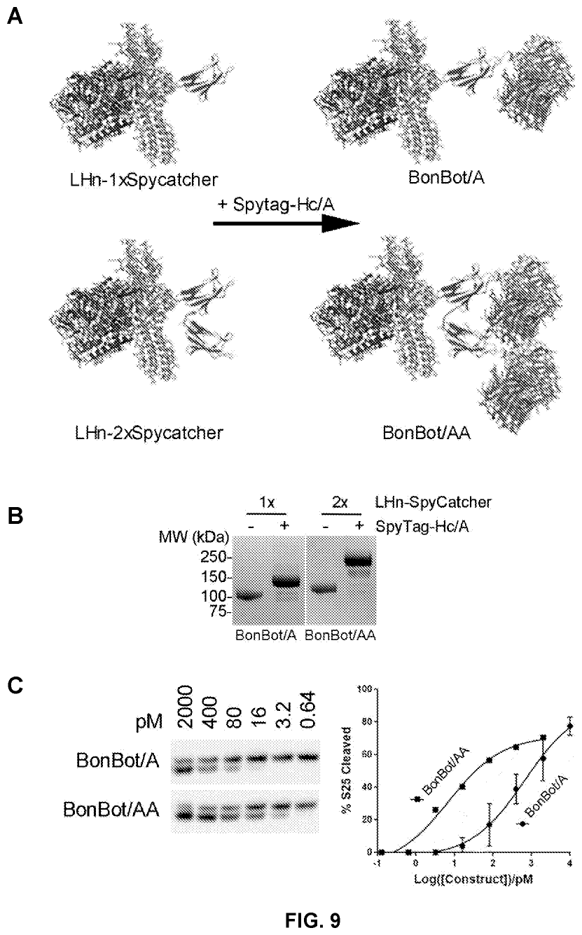

The disulphide-linked polypeptide described above carrying two cognate proteins, or two peptides, can also be used to construct novel highly efficient botulinum therapeutics incorporating alternative cell-binding elements. Indeed, disulphide-linked polypeptides carrying two binding domains were demonstrated to deliver botulinum enzyme into neurons with very high efficiency as demonstrated in FIG. 9 (see also [17]). Such cell-binding elements do not need to be botulinum neuronal binding domains. As would be clear to a person of skill in the art, botulinum enzymes can be targeted to cells using neuropeptides, antibodies and their fragments, lectins, growth factors, etc (see for example [22, 23]). Duplication and multiplication of cell-binding elements is well known to enhance cell-binding (see for example [24]). Duplication of binding domains is often observed in nature where high avidity is required for cell binding.

Accordingly, although the description provided herein focuses on neurotoxins comprising a SNARE peptidase domain, translocation domain and one or more neuronal binding domains, the invention applies equally to cell binding constructs that comprise a SNARE peptidase domain, translocation domain and one or more cell-binding elements such as one or more neuropeptides, antibodies and their fragments, lectins, growth factors. In this context, typically, the disulphide-linked polypeptide may comprise the SNARE peptidase domain and translocation domain (as described in detail elsewhere herein), which can then be covalently linked via one or more isopeptide bonds to one or more cell-binding elements using the peptide;cognate protein pairs described herein. Constructs comprising at least two cell-binding elements may therefore be generated by two isopeptide bonds forming between two peptide;cognate protein pairs (in a similar manner as is described above for constructs comprising two neuronal binding domains rather than two cell-binding elements). As would be clear to a person of skill in the art, in such examples, the terms “neuronal binding domain”, “third domain” and “fourth domain” may be replaced with “cell-binding element”. The terms “cell-binding element” and “cell binding domain” are used interchangeably herein.

The invention therefore also provides a cell binding construct comprising a SNARE peptidase domain, a translocation domain and a cell binding domain, wherein two of the domains are present within a disulphide-linked polypeptide that is covalently linked to the third domain via an isopeptide bond. The cell binding construct may comprise two cell binding domains. The disulphide-linked polypeptide may comprise the SNARE peptidase domain, the translocation domain and two cognate proteins, wherein each of the cognate proteins is covalently linked via an isopeptide bond to a corresponding peptide (which is linked to a cell binding domain). Alternatively, the disulphide-linked polypeptide may comprise the SNARE peptidase domain, the translocation domain and two peptides, wherein each of the peptides is covalently linked via an isopeptide bond to a corresponding cognate protein (which is linked to a cell binding domain). In this context, constructs may be generated comprising two identical or two distinct cell binding domains.

The common features of the above bonding systems include protein linking by a covalent chemical bond formed by two amino acids, more specifically by a covalent chemical bond formed by two amino acid side chains, more specifically by a chemical bond formed by two amino acid polar side chains, more specifically by a chemical bond formed by an amino acid basic side chain and one of these polar side chains: aspartate, glutamate, asparagine, glutamine, more specifically by a chemical bond formed by lysine and aspartate side chains, more specifically by a chemical bond formed by the positively charged amino group of a lysine side chain and the acidic side chain of an aspartate side chain such that two large proteins form spontaneous intramolecular isopeptide bonds.

As stated elsewhere herein, preferably, the SNARE peptidase domain and the translocation domain are from the same botulinum neurotoxin (as the SNARE peptidase domain and translocation domain of each neurotoxin are typically inseparable in terms of structure and function). By contrast, the neuronal binding domain may be the neuronal binding domain of any of the clostridial neurotoxins (i.e. botulinum neurotoxin type A, B, C, D, E, F, G, X or chimeric botulinum neurotoxins). By selecting specific neuronal binding domains, the resultant neurotoxin can be used to more effectively target specific neuronal populations.

Accordingly, in one example, the SNARE peptidase domain, translocation domain and neuronal binding domain of the neurotoxin are a botulinum neurotoxin SNARE peptidase domain, a botulinum neurotoxin translocation domain and a botulinum neurotoxin neuronal binding domain. In this example, the SNARE peptidase domain, translocation domain and neuronal binding domain may all be from the same botulinum type (i.e. type A, B, C, D, E, F, G, X or chimeric botulinum neurotoxins). Alternatively, the SNARE peptidase domain and translocation domain may be from the same botulinum type, with the neuronal binding domain being from a different botulinum type (or even from tetanus).

Accordingly, for example, the SNARE peptidase domain and translocation domain may both be botulinum type A, with the neuronal binding domain being botulinum type B. Alternatively, the SNARE peptidase domain and translocation domain may both be botulinum type A, with the neuronal binding domain being botulinum type C. Alternatively, the SNARE peptidase domain and translocation domain may both be botulinum type A, with the neuronal binding domain being botulinum type D. Alternatively, the SNARE peptidase domain and translocation domain may both be botulinum type A, with the neuronal binding domain being botulinum type E. Alternatively, the SNARE peptidase domain and translocation domain may both be botulinum type A, with the neuronal binding domain being botulinum type F. Alternatively, the SNARE peptidase domain and translocation domain may both be botulinum type A, with the neuronal binding domain being botulinum type G. Alternatively, the SNARE peptidase domain and translocation domain may both be botulinum type A, with the neuronal binding domain being botulinum type X.

In one example, the neuronal binding domain is botulinum type A (i.e. comprises the amino acid sequence of SEQ ID NO: 6, or a functional variant thereof as described elsewhere herein). In another example, the neuronal binding domain is botulinum type C (i.e. comprises the amino acid sequence of SEQ ID NO: 7, or a functional variant thereof as described elsewhere herein). This may be within the context of the SNARE peptidase domain and translocation domain both being botulinum type A.

The disulphide-linked polypeptide typically includes additional sequences to the two neurotoxin domains described herein. Similarly, the polypeptide comprising the third domain may have additional sequences (that are not part of the third domain itself). Such additional sequences may be referred to as extension sequences, spacer domains or spacer peptides. As used herein, a “spacer peptide” refers to a peptide sequence that is used to spatially separate two protein domains in the final neurotoxin structure/neurotoxin protein complex. The terms “spacer peptide”, “spacer domain”, “spacer peptide sequence” and “spacer peptide region” are used interchangeably herein.

Extension sequences or spacer peptides are particularly useful in the context of the invention to spatially separate the neurotoxin component parts of the disulphide-linked polypeptide from the third domain of the neurotoxin. Suitably, the neurotoxin component parts of the disulphide-linked polypeptide and the third domain of the neurotoxin may be spatially separated by e.g. by a distance of at least 4 nm e.g. at least 4.7 nm. Suitably, this distance may be further increased by using an extension sequence, for example a rigid trihelical extension. Inclusion of such extension sequences may increase the distance between the neurotoxin component parts of the disulphide-linked polypeptide and the third domain of the neurotoxin to, for example, at least 5 nm, at least 5.5 nm, at least 5.6 nm, at least 10 nm e.g. at least 10.3 nm etc.

In one example, the disulphide-linked polypeptide may typically comprise the SNARE peptidase domain and the translocation domain, which is then covalently linked to the neuronal binding domain via an isopeptide bond. Typically, in such embodiments, the SNARE peptidase domain is N-terminal to the translocation domain within the disulphide-linked polypeptide (such that the translocation domain is C-terminal to the SNARE peptidase domain). Typically, the disulphide-linked polypeptide is then N-terminal to the neuronal binding domain in the covalently linked neurotoxin. In such constructs, the covalent link is therefore between the translocation domain of the disulphide-linked polypeptide and the neuronal binding domain. The covalent link then spatially separates the translocation domain from the neuronal binding domain by a suitable distance e.g. by a distance of at least 4 nm e.g. at least 4.7 nm. Suitably, this distance may be further increased by using an extension sequence, such as a rigid trihelical extension. Inclusion of such extension sequences may increase the distance between the translocation domain and the neuronal binding domain to, for example, at least 5 nm, at least 5.5 nm, at least 5.6 nm, at least 10 nm e.g. at least 10.3 nm etc. Increasing the distance between the translocation domain and the neuronal binding domain is shown herein to be beneficial in preventing, regulating or reducing neuropathic pain and sweating.

In one example, the spacer peptide is a rigid or flexible spacer peptide. The spacer peptide may be located between the disulphide-linked polypeptide and the attached linking peptide/cognate protein. Alternatively, the spacer peptide may be located between the third domain and its attached linking peptide/conjugate protein. By inserting a spacer peptide of suitable length it is possible to generate neurotoxins wherein the neurotoxin domains were kept at a favourable distance from each other, with optimal bioactivity.

Flexible spacer peptides are usually applied when the joined domains require a certain degree of movement or interaction. Flexible spacer peptides in natural proteins can vary in length from 5 up to 20 amino acids and are generally composed of small, non-polar (e.g. Gly) or polar (e.g. Ser or Thr) amino acids. The small size of these amino acids provides flexibility, and allows for mobility of the connecting functional domains. The length of the flexible spacer peptides can be adjusted to allow for proper folding or to achieve optimal biological activity of the fusion proteins. Spacer peptides may be a longer (flexible) spacer peptide of any appropriate length, for example at least 25 amino acids in length, at least 30, at least 31, at least 35, at least 39, at least 40, at least 45, at least 50, at least 55, at least 60, at least 65, at least 66 amino acids in length (or any ranges therein between). In one embodiment, the spacer peptide is of 31 to 66 (e.g. 39 to 66) amino acids in length. Methods for determining the exact sequence of appropriate spacer peptides for use in the invention are well known in the art.

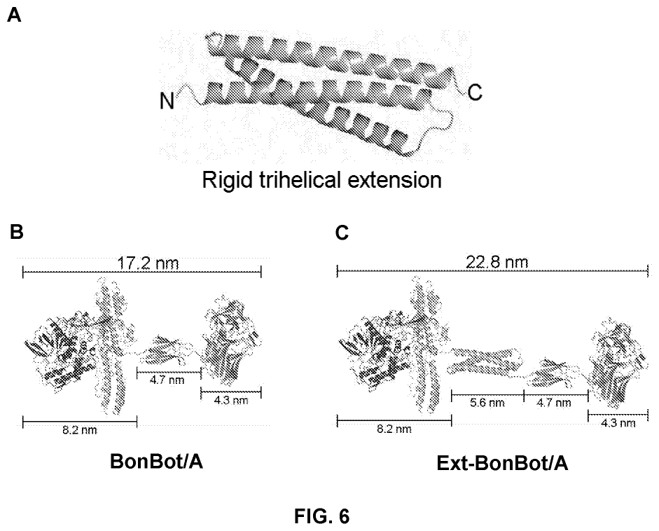

Rigid spacer peptides can also be used. They can for examples be created by using trihelical extensions where N- and C-termini are at the opposite end of a helical bundle (see for example FIGS. 3 and 6, which describes use of the rigid trihelical domain of syntaxin 1 in the neurotoxins described herein). Advantageously, this trihelical domain allows for addition of proteins specifically on opposite sides of the extension. The inventors have shown that inclusion of such rigid trihelical extensions within the neurotoxin can improve therapeutic properties e.g. reduce undesired paralytic properties of the neurotoxin. The rigid trihelical extension described herein provides an example of a polypeptide which can be used to alter the structure of a botulinum molecule to make it less paralysing. Use of such rigid trihelical extensions is therefore particularly preferable within the neurotoxins described herein, however, other sequences may also be used to the same effect.

A neurotoxin comprising a rigid trihelical extension as part of the disulphide linked polypeptide has been exemplified herein. The rigid trihelical extension increases the distance (spatial distance) between the domains within the disulphide linked polypeptide and the third domain. Typically, the disulphide linked polypeptide comprises its domains in the following order (from N-terminus to C-terminus): SNARE peptidase domain, translocation domain, rigid trihelical extension and a C-terminal cognate protein. The rigid trihelical extension thus increases the distance between the translocation domain of the disulphide linked polypeptide and the third domain of the resultant neurotoxin (the neuronal binding domain), which is covalently linked to the disulphide linked polypeptide via a peptide at its N terminal end.

As would be clear to a person of skill in the art, other arrangements of the component parts that achieve the desired distance between the translocation domain and the neuronal binding domain in the resultant neurotoxin may alternatively be used. For example, the rigid trihelical extension may be located with the third domain (the neuronal binding domain) rather than being part of the disulphide linked polypeptide. In this example, the disulphide linked polypeptide comprises its domains in the following order (from N-terminus to C-terminus): SNARE peptidase domain, translocation domain, and a C-terminal cognate protein; which is then covalently linked to the third domain via the following construct: peptide, rigid trihelical domain, neuronal binding domain.

As would also be clear to a person of skill in the art, the rigid trihelical domain described in the above examples may also be replaced by a different spacer sequence. Alternative spacer sequences that would be suitable in the context of the invention are described in more detail elsewhere herein.

Methods for generating the protein components (e.g. fusion proteins) described herein are well known in the art and include any suitable technique. Suitable techniques are readily identifiable by a person of skill in the art (e.g. cloning and growth in bacteria such as E. coli).

The neurotoxins described herein may be provided as part of a composition (e.g. a pharmaceutical composition). Such compositions may be used for therapeutic purposes as outlined below.

### Compositions

A neurotoxin as provided herein may be part of a composition (e.g. a pharmaceutical composition) that comprises the neurotoxin and one or more other components. A composition may be a composition that comprises a neurotoxin of the invention and a pharmaceutically acceptable excipient, adjuvant, diluent and/or carrier. Compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected neurotoxin without causing any undesirable biological effects or interacting in a deleterious manner with any of the other components of the pharmaceutical composition in which it is contained.

Excipients are natural or synthetic substances formulated alongside an active ingredient (e.g. a neurotoxin as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art and include cell-penetrating peptides. A suitable adjuvant is therefore easily identifiable by one of ordinary skill in the art.

Diluents are diluting agents. Pharmaceutically acceptable diluents are well known in the art and include water or saline. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art and include serum albumin. A suitable carrier is therefore easily identifiable by one of ordinary skill in the art.

### Treatment of a Subject

The compositions provided herein may be used for therapeutic purposes as outlined below. A composition of the invention is particularly advantageous for preventing, regulating or reducing neuropathic pain or sweating in a subject.

Alternatively, or additionally, a composition of the invention may be used for preventing, regulating or reducing sweating due to excessive neuronal activity in a subject. In this context “excessive neuronal activity” refers to an increase in neuronal activity compared to the norm. Examples of excessive neuronal activity that may result in sweating that may be prevented, reduced or regulated using a composition of the invention include focal hyperhidrosis of the palms, armpits and/or soles.

Advantageously, compositions of the invention may also be used for therapeutic purposes. As used herein, the term “therapeutic” is intended to refer to “treatment” of a subject.

As used herein, the terms “treat”, “treating” and “treatment” are taken to include an intervention performed with the intention of preventing the development or altering the pathology of a condition, disorder or symptom. Accordingly, “treatment” refers to both therapeutic treatment and prophylactic or preventative measures, wherein the object is to prevent or slow down (lessen) the targeted condition, disorder or symptom. As used herein, the terms “disease” and “disorder” are used interchangeably.

As used here in the term “subject” refers to an individual, e.g., a human, dog, cat, pig, horse, mouse, cow, rat etc having or at risk of having a specified condition, disorder or symptom. The subject may be a patient i.e. a subject in need of treatment in accordance with the invention. The subject may have received treatment for the condition, disorder or symptom. Alternatively, the subject has not been treated prior to treatment in accordance with the present invention.

Compositions provided herein may be used for treating or preventing a condition, disorder or symptom which is alleviated by the inhibition of neural terminals.

The skilled person will be fully aware of the diseases or conditions which are alleviated by the inhibition of neural terminals since the use of botulinum toxin A has been in widespread use for medicinal and cosmetic therapies for a number of years (see, for example, [8]). In particular, some of the diseases or conditions which are alleviated by the inhibition of neural terminals are selected from the group consisting of: pain, migraine, chronic tension headaches, excessive sweating, salivation, gastrointestinal disorders, urinary disorders, hyperlacrymation, hyperhidrosis.

Accordingly, in one aspect, the neurotoxins described herein are particularly useful in preventing or treating pain, migraine, chronic tension headaches, excessive sweating, salivation, gastrointestinal disorders, urinary disorders, hyperlacrymation and hyperhidrosis.

A well-known impediment regarding the treatment of migraine with commercially available BoTox is that it causes muscle paralysis. The reduced paralytic properties of BonBots can be advantageous for treatment of migraine and other conditions.

Compositions of the invention may therefore be used to treat or prevent a neurological condition, disorder or symptom in a subject. Methods of treating or preventing a neurological condition, disorder or symptom are also provided, comprising administering a composition of the invention to a subject. Accordingly, in vivo methods of treatment are provided, which may be prophylactic and/or therapeutic.

As used herein, treating or preventing a “neurological condition, disorder and/or symptom” is intended to include treating or preventing secretions, pain, a neurological disorder or condition in a subject due to excessive functions.

In one embodiment, the pain is selected from the group consisting of: pain associated with neuromuscular disorders, pain associated with arthritis, pain associated with trigeminal neuralgia, headache pain, inflammatory nociceptive pain, and neuropathic pain.

In one embodiment, the neuropathic pain is selected from the group consisting of: cancer pain, post-operative neuropathic pain, allodynia, post-herpetic neuralgia bone pain, peripheral neuropathy.

In one embodiment, the cholinergic controlled secretion is selected from the group consisting of: lacrimation, salivation, mucus secretion, gastrointestinal secretion and hyperhidrosis.

To date the benefits of botulinum neurotoxins have been restricted to treatments of neuromuscular conditions and disorders of the peripheral nervous system. Botulinum neurotoxins, however, can also block neurotransmitter release in central neurons, making it possible to exploit them in experimental neuroscience and in future neurology dealing with higher brain functions. Studies on brain slices, cultured neurons and synaptosomes have demonstrated that botulinum neurotoxins can stop the neurotransmitter release of not only acetylcholine but also glutamate, glycine, noradrenaline, dopamine, serotonin, ATP and various neuropeptides [25-28].

The compositions described herein can be administered to the subject by any conventional route, including injection or by gradual infusion over time. The administration may, for example, be topical, intramuscular, intravascular, intracavity, intracerebral, intralesional, rectal, subcutaneous, transdermal, epidural, intrathecal, percutaneous, or by infusion.

The compositions described herein may be in any form suitable for the above modes of administration. For example, suitable forms for parenteral injection (including, intradermal, subcutaneous, intramuscular, infusion) include a sterile solution, suspension or emulsion; suitable forms for topical administration include microneedling, electrophoresis, ointment or cream. Alternatively, the route of administration may be by direct injection into the target area, or by regional delivery or by local delivery.

The compositions described herein are for administration in an effective amount. An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the patient/subject. For example, the suitable dosage of the neurotoxin for a given patient/subject will be determined by the attending physician (or person administering the composition), taking into consideration various factors known to modify the action of the neurotoxin for example severity and type of disorder, condition or symptom, body weight, sex, diet, time and route of administration, other medications and other relevant clinical factors. The dosages and schedules may be varied according to the particular condition, disorder or symptom the overall condition of the patient/subject. Effective dosages may be determined by either in vitro or in vivo methods.

The compositions of the present invention are advantageously presented in unit dosage form e.g. nanograms.

### Methods of Producing Neurotoxins

A method of producing a neurotoxin comprising a SNARE peptidase domain, a translocation domain and a neuronal binding domain is also provided, the method comprising mixing a disulphide-linked polypeptide comprising two of the domains with a polypeptide comprising the third domain, wherein

(i) a peptide is attached to the disulphide-linked polypeptide and a cognate protein is attached to the third domain; or

(ii) a cognate protein attached to the disulphide-linked polypeptide and a peptide is attached to the third domain,

such that, on mixing, an isopeptide bond is formed between the peptide and its cognate protein to covalently link the disulphide-linked polypeptide to the third domain.

The disulphide-linked polypeptide comprising two of the domains may be mixed with the polypeptide comprising the third domain in any appropriate buffer for example HEPES or a phosphate buffer. Salt may also be present. For example, an appropriate buffer of 20 mM HEPES, pH 7.3, 100 mM NaCl may be used. Mixing may occur for at least one hour, e.g. at least 2 hours, at least 3 hours, at least 4 hours, up to 24 hours etc until the neurotoxin has been produced. A non-limiting example of the reagents and protocol that may be used is provided in the examples section below, however, as would be clear to a person of skill in the art, routine experimentation of suitable conditions and reagents is possible to optimise the method and/or identify alternative reagents and/or protocols that are also suitable for performing the methods described herein.

### Kits

A kit is also provided for producing a neurotoxin comprising a SNARE peptidase domain, a translocation domain and a neuronal binding domain, the kit comprising:

(a) a disulphide-linked polypeptide comprising two of the domains; and

(b) a polypeptide comprising the third domain, wherein
(i) a peptide is attached to the disulphide-linked polypeptide and a cognate protein is attached to the third domain; or
(ii) a cognate protein attached to the disulphide-linked polypeptide and a peptide is attached to the third domain,

such that, on mixing, an isopeptide bond is formed between the peptide and its cognate protein to covalently link the disulphide-linked polypeptide to the third domain.

The kits described herein may also include an appropriate buffer in which the polypeptides of (a) and (b) could be mixed. A suitable buffer may be a HEPES or phosphate buffer. Salt may also be present within the buffer. For example, an appropriate buffer of 20 mM HEPES, pH 7.3, 100 mM NaCl may be present within the kit.

### General Definitions

As used herein, a “naturally-occurring” polypeptide refers to an amino acid sequence that occurs in nature.

A “non-essential” (or “non-critical”) amino acid residue is a residue that can be altered from the wild-type sequence of (e.g., the sequence of SEQ ID NOs:1 to 22) without abolishing or, more preferably, without substantially altering a biological activity, whereas an “essential” (or “critical”) amino acid residue results in such a change. For example, amino acid residues that are conserved are predicted to be particularly non-amenable to alteration, except that amino acid residues within the hydrophobic core of domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential (or non-critical) amino acid residue in a protein is preferably replaced with another amino acid residue from the same side chain family. Alternatively, in another embodiment, mutations can be introduced randomly, and the resultant mutants can be screened for biological activity to identify mutants that retain activity.

As used herein, a “biologically active portion” of protein or a protein portion with “biological activity” includes a fragment of protein that participates in an interaction between molecules and non-molecules. Biologically active portions of proteins include peptides comprising amino acid sequences sufficiently homologous to or derived from the amino acid sequences of the protein, e.g., the amino acid sequences shown in SEQ ID NO: 1 to 22, which include fewer amino acids than the full length protein, and exhibit at least one activity of the encoded protein. Typically, biologically active portions comprise a domain or motif with at least one activity of the protein, e.g., the biologically active portion may retain one of the following activities (as appropriate); Bot/Nbd type A activity, Bot/Nbd type B activity, Bot/Nbd type C activity, Bot/Nbd type D activity, Bot/Nbd type E activity, Bot/Nbd type F activity or Bot/Nbd type G activity. In this context, “activity” is used to mean the functional activity of each binding domain (i.e. its binding capacity).

A biologically active portion of protein can be a polypeptide that is, for example, 50, 100, 150, 200, 250, 300, 350, 400, 450, 500 or more amino acids in length of SEQ ID NO:1 to 22.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970, J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989, CAB/OS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990, J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

The polypeptides described herein can have amino acid sequences sufficiently or substantially identical to the amino acid sequences of SEQ ID NO:1 to 22. The terms “sufficiently identical” or “substantially identical” are used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Explanation of Abbreviations Used

As used herein, the term “LHn/A” refers to a disulphide-linked polypeptide comprising a botulinum neurotoxin type A SNARE peptidase domain and a botulinum neurotoxin type A translocation domain. The term “LHn/A-Spycatcher” refers to a fusion protein comprising (N to C-terminal): a botulinum neurotoxin type A SNARE peptidase domain, a botulinum neurotoxin type A translocation domain; and Spycatcher. Furthermore, the term “LHn/A-ext-Spycatcher” refers to a fusion protein comprising (N to C-terminal): a botulinum neurotoxin type A SNARE peptidase domain, a botulinum neurotoxin type A translocation domain with a rigid extension domain, and Spycatcher.

As used herein, the term “Hc/A” refers to a botulinum neurotoxin type A neuronal binding domain. The term “Spytag-Hc/A” refers to a fusion protein comprising (N to C-terminal): Spytag and a botulinum neurotoxin type A neuronal binding domain.

As used herein, the term “botulinum neurotoxin” and its abbreviation “bot” are used interchangeably. For example, botulinum neurotoxin type A may also be referred to herein as “botulinum neurotoxin A” or “bot/A”. The novel botulinum neurotoxins provided herein are referred to as “Bonded Botulinum” or “BonBot” (e.g. BonBot/A). For the avoidance of doubt, BonBot/A (i.e. a neurotoxin of the invention made up of a botulinum neurotoxin type A SNARE peptidase domain, a botulinum neurotoxin type A translocation domain and a botulinum neurotoxin type A neuronal binding domain wherein two of the domains are present within a disulphide-linked polypeptide that is covalently linked to the third domain via an isopeptide bond) is also referred to herein as “BonBot/A”. Furthermore, the term “ext-BonBot/A” refers to a modified form of bonbot/A wherein the neurotoxin type A translocation domain has a rigid extension domain.

The term “BonBot/C” refers to a bonded neurotoxin of the invention made up of a botulinum neurotoxin type A SNARE peptidase domain, a botulinum neurotoxin type A translocation domain and a botulinum neurotoxin type C neuronal binding domain (wherein the SNARE peptidase domain and translocation domain are present within a disulphide-linked polypeptide that is covalently linked to the neuronal binding domain via an isopeptide bond; e.g. using the Spycatcher:Spytag bonding system).

### Example 1: Use of Spycatcher Technology for Making Botulinum Molecules

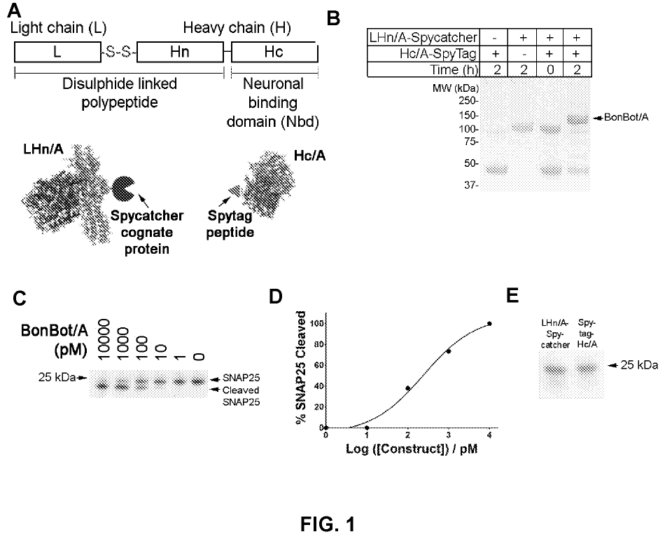

The inventors have produced functional botulinum neuronal modulator (referred to as BonBot/A herein) using the Spycatcher bonding system (FIG. 1). They fused the Light chain/translocation part of Bot/A (LHn/A, aa 1-873) to Spycatcher (LHn/A-Spycatcher). Note, the LHn/A protein carries LVPRGS sequence, which is nicked by Thrombin during protein purification; this nicking is important for the activation of the botulinum enzymatic part as described in a 2010 publication [14]. They then fused the neuronal binding domain of Bot/A (Hc/A, aa 874-1296) to Spytag (Spytag-Hc/A).

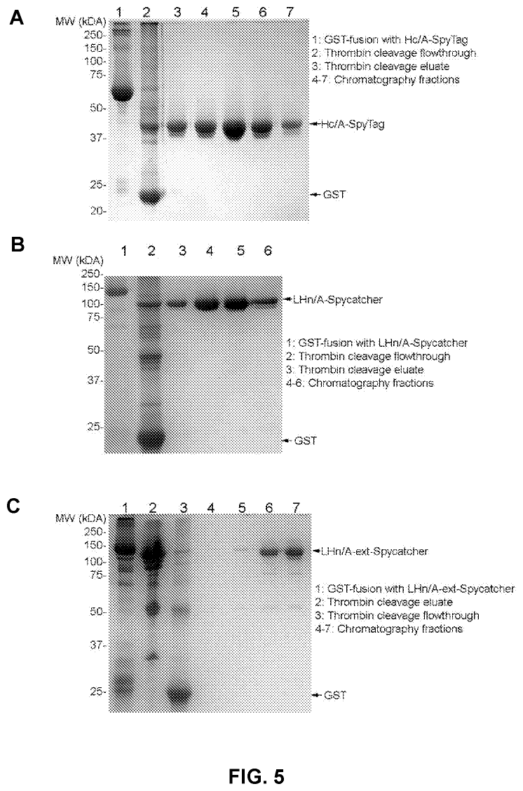

Both proteins were produced in E. coli bacteria as Glutathione-S-transferase (GST) fusion proteins and purified on glutathione-Sepharose beads. The two proteins were freed from the beads using Thrombin treatment with further purification by size-exclusion chromatography on Superdex-200 column equilibrated in 100 mM NaCl, 20 mM Hepes, pH 7.3 (FIG. 5). Following purification, the proteins were aliquoted and stored in −80° C. freezer.

The bonding of separately produced Bot/A parts was evaluated by SDS-polyacrylamide electrophoresis (SDS-PAGE). The two parts were mixed at 22° C. and following 2 hour incubation, an SDS sample buffer was added with subsequent 5 min boiling. Under such harsh conditions, protein complexes normally disintegrate, but covalently bonded proteins will stay together. SDS-PAGE gel was stained by Coomassie stain revealing the bonding of two botulinum parts. FIG. 1B shows that the 2-hour incubation of the two botulinum parts is sufficient to produce a high molecular weight protein (Bonded Botulinum A or BonBot/A) which is the sum of LHn-Spycatcher and Hc-Spytag.

BonBot/A was evaluated for its biological activity. Native Bot/A causes nerve block by cleaving an intraneuronal protein called SNAP25. This can be visualised in differentiated neuroblastoma cell cultures as a shift in molecular weight of the SNAP25 protein. The inventors incubated differentiated SiMa human neuroblastoma cells with the BonBot/A for 48 hours to allow binding, translocation of the botulinum enzyme, and catalytic cleavage of SNAP25 within the neuroblastoma cells. FIG. 1C, D shows that BonBot/A cleaved SNAP25 within 48 hours, in picomolar range whereas the individual two botulinum parts were without any effect on SNAP25 even at nanomolar concentrations (FIG. 1E). This shows that the two parts need to be linked together before they can exert their complementary functions on neurons: binding, cytosol penetration and cleavage of SNAP25. The efficiency of cleavage was approx. 10 times less than that of the native Bot/A.

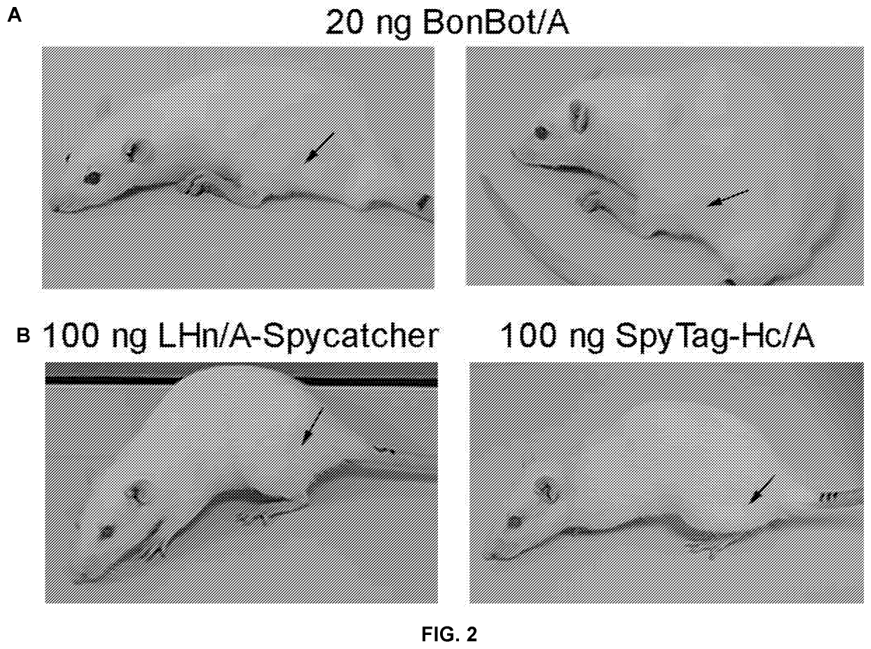

BonBot/A (20 ng) was injected into the gastrocnemius muscle of adult rats. Following 48 hrs period, a characteristic splaying of the injected leg indicating local paralysis was observed without any other signs of physiological distress (FIG. 2A). The efficiency of muscle paralysis is less than that of the native Bot/A, since the half-lethal dose LD50 for the native Bot/A in rats is less than 2 nanograms. To rule out non-specific behaviour of botulinum parts, the two individual proteins, LHn/A-Spycatcher and Spytag Hc/A, were analysed separately on muscle paralysis. Injections of either 100 ng LHn/A-Spycatcher or 100 ng Spytag Hc/A into the gastrocnemius muscle did not cause any changes in the motor behaviour of rats (FIG. 2B). The rats injected with individual proteins moved normally post-injection. This shows that the two parts need to be linked together before they can exert their complementary functions on neurons: binding, cytosol penetration and cleavage of SNAP25.

The reduced paralytic ability of BonBot/A is likely due to the structural extension of the botulinum neurotoxin due to the Spycatcher system. The inventors explored if a further extension could cause reduction in muscle paralysis. LHn/A carrying an extension followed by the Spycatcher sequence was prepared (FIG. 3; LHn/A-extension-Spycatcher). The extension is a rigid trihelical syntaxin fragment with molecular weight of 15 kDa and 5.6 nm in length (FIG. 6) [29]. When mixed with Spytag-Hc/A, the LHn/A-extension-Spycatcher transitioned into the new botulinum molecule named extended (ext-) BonBot/A. Injections of 100 ng of ext-BonBot/A into the gastrocnemius muscle did not cause paralysis, confirming that step-by-step structural extensions can reduce activity of botulinum molecules on neuromuscular junctions.

The inventors further investigated the paralytic activity of bonded Bitoxes using electromyography (EMG) to allow direct assessment of individual muscle function following subcutaneous injections. Lightly anaesthetized rats were injected subcutaneously with 2 ng BonBot/A or extended ext-BonBot/A above the gastrocnemius muscle and muscle activity in response to an electrical stimulation was recorded. Stimulating electrodes were inserted into the plantar surface of the paw and current passed through in order to elicit a withdrawal. Recording electrodes across the gastrocnemius muscle measured the voltage of the muscle contraction as an EMG signal to ascertain whether any paralysis occurred due to injection of toxin. Baseline EMG measurements were recorded on day 0 and whilst the animal remained under anesthesia, 2 ng/30 μl of toxin (n=4 per group) was administered subcutaneously above the gastrocnemius muscle. Animals were observed to full recovery and returned to their home cage. Subsequent EMG measurements were recorded on days 1, 2, 3 and 7. Quantitative analysis of the EMG data revealed that animals from the BonBot/A group exhibited motor deficit whereas the ext-BonBot/A motor function did not differ significantly from the baseline values (FIG. 3E).

### Example 2: Botulinum Neurotoxin Chimeras

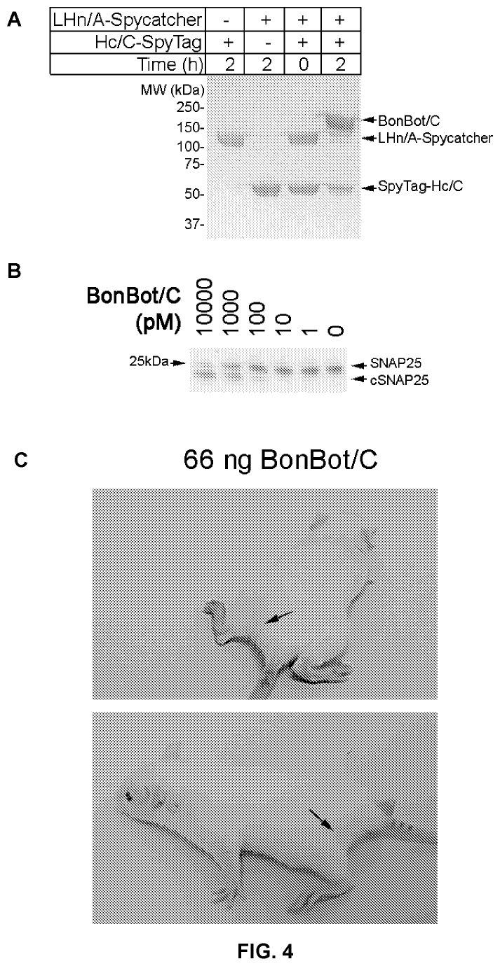

The inventors also made a new chimera wherein LHn type A is bonded to Spytag-Hc type C. FIG. 4A shows that simple mixing of LHn/A-Spycatcher with Spytag-Hc type C bonds the two proteins into a high molecular weight protein BonBot/C as evidenced by migration of the latter in the SDS-PAGE gel. Whether BonBot/C is active in neuronal cells was tested. Western immunoblotting demonstrated that BonBot/C was capable of cleaving SNAP25 in neuronally-differentiated Sima neuroblastoma cells (FIG. 4B). The inventors evaluated BonBot/C on motor paralysis. 13, 33 and 66 ng were injected into the gastrocnemius muscle of 4 weeks old rats. FIG. 5 shows that BonBot/C caused muscle paralysis evidenced by leg splaying only at 66 ng, indicating a different neuromuscular activity compared to BonBot/A. No muscle paralysis was observed with 13 and 33 ng after 3 days post-injection.

### Example 3: Therapeutic Effects of Bonded Botulinum

The therapeutic effects of extended Bonded Botulinum (ext-BonBot/A) in the Spared nerve injury (SNI) model of neuropathic pain was evaluated using subdermal intraplantar injections.

### Rat Model of Neuropathic Pain.

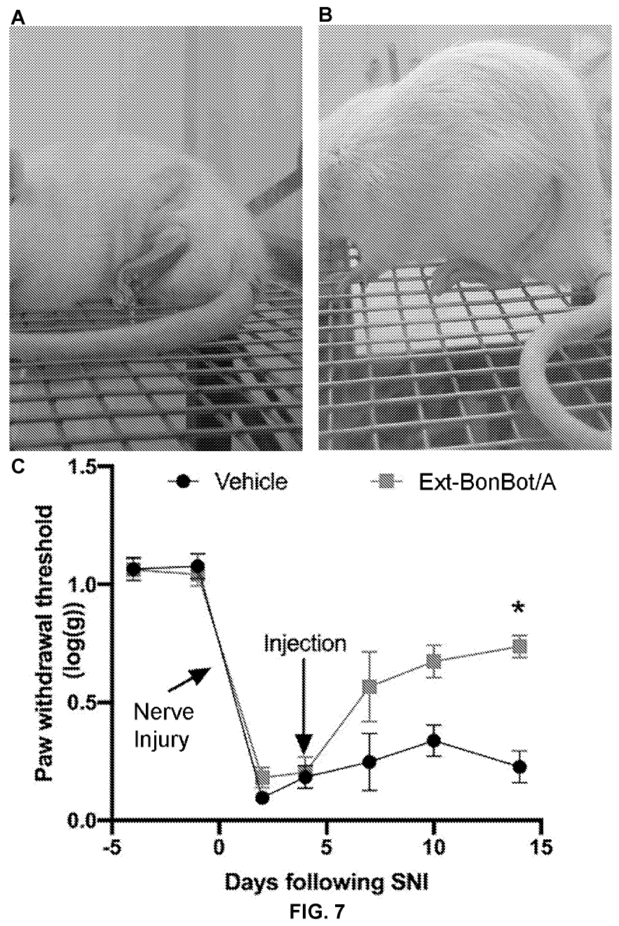

Male Sprague Dawley rats were anaesthetised and the sciatic nerve exposed. The tibial and common peroneal branches were tightly ligated using 5/0 silk and sectioned to leave the sural nerve intact. Behavioural paw withdrawal was analysed to ascertain sensory disturbances using von Frey mechanical sensitivity tests. The investigators were blind to the treatment group. Baseline measurements were undertaken 4 and 1 day before nerve injury. Following habituation to the behavioural apparatus, animals were stimulated using a series of calibrated von Frey filaments via the lateral plantar surface of the paw. The threshold was determined using the Up-Down method and the 50% paw withdrawal threshold calculated. Four days following spared nerve injury (SNI) animals underwent sub-dermal injection of either a vehicle (n=4) or ext-BonBot/A (10 ng) (n=4) into the plantar surface of the ipsilateral paw. Changes in animal behaviour and gait were observed following SNI and sub-plantar injection of either vehicle or Bonded Botulinum (10 ng/30 μl). Animals injected with vehicle showed reluctance to place ipsilateral hindpaw onto the mesh surface and showed reduced weight bearing on the affected limb (FIG. 7A). In contrast, animals injected with Bonded Botulinum more readily made contact with the mesh flooring and showed more normal gait behaviour (FIG. 7B). Animals having been injected with ext-BonBot/A exhibited a functional recovery (FIG. 7C) and reduced pain-like symptoms placing the affected leg on the surface of the mesh cage more readily. Unexpectedly, these animals also showed much dryer plantar skin on the ipsilateral paw compared with the animals receiving vehicle treatment.

### Mouse Model of Neuropathic Pain.

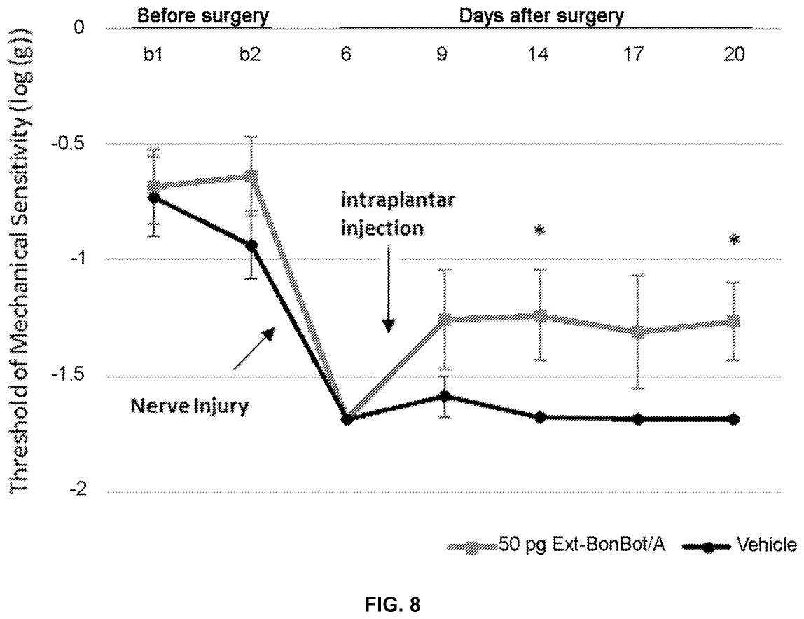

The spared nerve injury (SNI) was performed also in adult mice. Briefly, under isoflurane anaesthesia the skin on the lateral surface of the thigh was incised and a section made directly though the biceps femoris muscle exposing the sciatic nerve and its three terminal branches: the sural, the common peroneal and the tibial nerves. The common peroneal and the tibial nerves were tight-ligated with 5-0 silk and sectioned distal to the ligation. Great care was taken to avoid any contact with the spared sural nerve. Complete haemostasis was confirmed and the wound was sutured. Mechanical thresholds were assessed using Von Frey filaments in mice before SNI, after SNI and after ext-BonBot/A (50 pg in 20 microliters) was injected 5 days after SNI surgery. Animals were placed in Plexiglas chambers, located on an elevated wire grid, and allowed to habituate for at least 1 hour. After this time, the plantar surface of the paw was stimulated with a series of calibrated Von Frey's monofilaments. The threshold was determined by using the Up-Down method. The data were expressed as log of the mean of the 50% pain threshold±SEM. FIG. 8 shows that similar to rats, ext-BonBot/A caused a sustained reduction in mechanical hypersensitivity after nerve injury even at the picogram doses.

### Example 4: Use of Spycatcher Technology for Making Larger, More Efficient Botulinum Molecules—Duplication of Botulinum Parts

The inventors made a variant of the LHn-Spycatcher molecule, wherein LHn type A is recombinantly fused to two sequential copies of Spycatcher (LHn-2×Spycatcher, SEQ ID NO: 31) allowing for production of botulinum molecules containing 2 binding domains as opposed to botulinum molecules with a single binding domain (FIG. 9A). The term “LHn/A-2×Spycatcher” refers to a fusion protein comprising (N to C-terminal): a botulinum neurotoxin type A SNARE peptidase domain, a botulinum neurotoxin type A translocation domain, first Spycatcher; and second copy of Spycatcher. FIG. 9B shows that simple mixing of LHn/A-2×Spycatcher with Spytag-Hc/A bonds polypeptides into a protein, BonBot/AA, that carries two binding domains and is roughly 50 kDa higher in molecular weight than BonBot/A, as evidenced by migration of the latter in the SDS-PAGE gel. Activity of BonBot/AA was tested in neuronal cell cultures. Western immunoblotting demonstrated that BonBot/AA was 30 times more active in cleaving SNAP25 in neuronally-differentiated human neuroblastoma cells compared to BonBot/A with single binding domain (FIG. 9C).

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

SEQUENCES
Spytag amino acid sequence (SEQ ID NO: 1):
AHIVMVDAYKP
Spycatcher amino acid sequence (SEQ ID NO: 2):
AMVDTLSGLSSEQGQSGDMTIEEDSATHIKFSKRDEDGKELAGATMELRDSSGKTISTWIS
DGQVKDFYLYPGKYTFVETAAPDGYEVATAITFTVNEQGQVTVNGKATKGDLE
Botulinum neurotoxin type A (1-448) SNARE peptidase domain
amino acid sequence (SEQ ID NO: 3):
MPFVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGSTIDT
ELKVIDTNCINVIQPDGSYRSEELNLVIIGPSADIIQFECKSFGHEVLNLTRNGYGSTQYIRFSP
DFTFGFEESLEVDTNPLLGAGKFATDPAVTLAHELIHAGHRLYGIAINPNRVFKVNTNAYYE
MSGLEVSFEELRTFGGHDAKFIDSLQENEFRLYYYNKFKDIASTLNKAKSIVGTTASLQYMK
NVFKEKYLLSEDTSGKFSVDKLKFDKLYKMLTEIYTEDNFVKFFKVLNRKTYLNFDKAVFKINI
VPKVNYTIYDGFNLRNTNLAANFNGQNTEINNMNFTKLKNFTGLFEFYKLLCVRGIITSKTKS
LDKGYNK
Botulinum neurotoxin type A (449-874) translocation domain
amino acid sequence (SEQ ID NO: 4):
QALNDLCIKVNNWDLFFSPSEDNFTNDLNKGEEITSDTNIEAAEENISLDLIQQYYLTFNFDN
EPENISIENLSSDIIGQLELMPNIERFPNGKKYELDKYTMFHYLRAQEFEHGKSRIALTNSVNE
ALLNPSRVYTFFSSDYVKKVNKATEAAMFLGWVEQLVYDFTDETSEVSTTDKIADITIIIPYIG
PALNIGNMLYKDDFVGALIFSGAVILLEFIPEIAIPVLGTFALVSYIANKVLTVQTIDNALSKRNE
KWDEVYKYIVTNWLAKVNTQIDLIRKKMKEALENQAEATKAIINYQYNQYTEEEKNNINFNID
DLSSKLNESINKAMININKFLNQCSVSYLMNSMIPYGVKRLEDFDASLKDALLKYIYDNRGTLI
GQVDRLKDKVNNTLSTDIPFQLSKYVDNQRLLSTFTEYIKNI
Ext-Bot/A translocation domain amino acid sequence for E. coli
expression (Thrombin-cleavinq site is underlined, extension in
bold, addition caused by recombinant fusion is in italics)
(SEQ ID NO: 5):
AKSLVPRGSQALNDLCIKVNNWDLFFSPSEDNFTNDLNKGEEITSDTNIEAAEENISLDLIQQ
YYLTFNFDNEPENISIENLSSDIIGQLELMPNIERFPNGKKYELDKYTMFHYLRAQEFEHGKS
RIALTNSVNEALLNPSRVYTFFSSDYVKKVNKATEAAMFLGWVEQLVYDFTDETSEVSTTDK
IADITIIIPYIGPALNIGNMLYKDDFVGALIFSGAVILLEFIPEIAIPVLGTFALVSYIANKVLTVQTI
DNALSKRNEKWDEVYKYIVTNWLAKVNTQIDLIRKKMKEALENQAEATKAIINYQYNQYTEE
EKNNINFNIDDLSSKLNESINKAMININKFLNQCSVSYLMNSMIPYGVKRLEDFDASLKDALLK
YIYDNRGTLIGQVDRLKDKVNNTLSTDIPFQLSKYVDNQRLLSTFTEYIKNIRSGILDSMGKD
RTQELRTAKDSDDDDDVTVTVDRDRFMDEFFEQVEEIRGFIDKIAENVEEVKRKHSAILAS
PNPDEKTKEELEELMSDIKKTANKVRSKLKSIEQSIEQEEGLNRSSADLRIRKTQHSTLSR
KFVEVMSEYNATQSDYRERCKGRIQRQLEITGRTTSMGGSGSGSGAMVDTLSGLSSEQG
QSGDMTIEEDSATHIKFSKRDEDGKELAGATMELRDSSGKTISTWISDGQVKDFYLYPGKY
TFVETAAPDGYEVATAITFTVNEQGQVTVNGKATKGDLE
Botulinum A (874-1296) neuronal bindinq domain amino acid sequence
(SEQ ID NO: 6):
INTSILNLRYESNHLIDLSRYASKINIGSKVNFDPIDKNQIQLFNLESSKIEVILKNAIVYNSMYE
NFSTSFWIRIPKYFNSISLNNEYTIINCMENNSGWKVSLNYGEIIWTLQDTQEIKQRVVFKYS
QMINISDYINRWIFVTITNNRLNNSKIYINGRLIDQKPISNLGNIHASNNIMFKLDGCRDTHRYI
WIKYFNLFDKELNEKEIKDLYDNQSNSGILKDFWGDYLQYDKPYYMLNLYDPNKYVDVNNV
GIRGYMYLKGPRGSVMTTNIYLNSSLYRGTKFIIKKYASGNKDNIVRNNDRVYINVVVKNKEY
RLATNASQAGVEKILSALEIPDVGNLSQVVVMKSKNDQGITNKCKMNLQDNNGNDIGFIGFH
QFNNIAKLVASNWYNRQIERSSRTLGCSWEFIPVDDGWGERPL
Botulinum C (872-1291) neuronal binding domain amino acid sequence
(SEQ ID NO: 7):
NDSKILSLQNRKNTLVDTSGYNAEVSEEGDVQLNPIFPFDFKLGSSGEDRGKVIVTQNENIV
YNSMYESFSISFWIRINKWVSNLPGYTIIDSVKNNSGWSIGIISNFLVFTLKQNEDSEQSINFS
YDISNNAPGYNKWFFVTVTNNMMGNMKIYINGKLIDTIKVKELTGINFSKTITFEINKIPDTGLI
TSDSDNINMWIRDFYIFAKELDGKDINILFNSLQYTNVVKDYWGNDLRYNKEYYMVNIDYLN
RYMYANSRQIVFNTRRNNNDFNEGYKIIIKRIRGNTNDTRVRGGDILYFDMTINNKAYNLFMK
NETMYADNHSTEDIYAIGLREQTKDINDNIIFQIQPMNNTYYYASQIFKSNFNGENISGICSIGT
YRFRLGGDWYRHNYLVPTVKQGNYASLLESTSTHWGFVPVSE
Botulinum B (862-1291) neuronal binding domain amino acid seguence
(SEQ ID NO: 8):
NNIILNLRYKDNNLIDLSGYGAKVEVYDGVELNDKNQFKLTSSANSKIRVTQNQNIIFNSVFLD
FSVSFWIRIPKYKNDGIQNYIHNEYTIINCMKNNSGWKISIRGNRIIWTLIDINGKTKSVFFEYNI
REDISEYINRWFFVTITNNLNNAKIYINGKLESNTDIKDIREVIANGEIIFKLDGDIDRTQFIWMK
YFSIFNTELSQSNIEERYKIQSYSEYLKDFWGNPLMYNKEYYMFNAGNKNSYIKLKKDSPVG
EILTRSKYNQNSKYINYRDLYIGEKFIIRRKSNSQSINDDIVRKEDYIYLDFFNLNQEWRVYTY
KYFKKEEEKLFLAPISDSDEFYNTIQIKEYDEQPTYSCQLLFKKDEESTDEIGLIGIHRFYESGI
VFEEYKDYFCISKWYLKEVKRKPYNLKLGCNWQFIPKDEGWTE
Botulinum D (865-1275) neuronal binding domain amino acid seguence
(SEQ ID NO: 9):
NDSKILSLQNKKNALVDTSGYNAEVRVGDNVQLNTIYTNDFKLSSSGDKIIVNLNNNILYSAIY
ENSSVSFWIKISKDLTNSHNEYTIINSIEQNSGWKLCIRNGNIEWILQDVNRKYKSLIFDYSES
LSHTGYTNKWFFVTITNNIMGYMKLYINGELKQSQKIEDLDEVKLDKTIVFGIDENIDENQML
WIRDFNIFSKELSNEDINIVYEGQILRNVIKDYWGNPLKFDTEYYIINDNYIDRYIAPESNVLVL
VQYPDRSKLYTGNPITIKSVSDKNPYSRILNGDNIILHMLYNSRKYMIIRDTDTIYATQGGECS
QNCVYALKLQSNLGNYGIGIFSIKNIVSKNKYCSQIFSSFRENTMLLADIYKPWRFSFKNAYT
PVAVTNYETKLLSTSSFWKFISRDPGWVE
Botulinum E (853-1251) neuronal binding domain amino acid seguence
(SEQ ID NO: 10):
SSVLNMRYKNDKYVDTSGYDSNININGDVYKYPTNKNQFGIYNDKLSEVNISQNDYIIYDNK
YKNFSISFWVRIPNYDNKIVNVNNEYTIINCMRDNNSGWKVSLNHNEIIWTLQDNAGINQKLA
FNYGNANGISDYINKWIFVTITNDRLGDSKLYINGNLIDQKSILNLGNIHVSDNILFKIVNCSYT
RYIGIRYFNIFDKELDETEIQTLYSNEPNTNILKDFWGNYLLYDKEYYLLNVLKPNNFIDRRKD
STLSINNIRSTILLANRLYSGIKVKIQRVNNSSTNDNLVRKNDQVYINFVASKTHLFPLYADTA
TTNKEKTIKISSSGNRFNQVVVMNSVGNNCTMNFKNNNGNNIGLLGFKADTVVASTWYYTH
MRDHTNSNGCFWNFISEEHGWQE
Botulinum F (866-1274) neuronal binding domain amino acid sequence
(SEQ ID NO: 11):
KDSSILDMRYENNKFIDISGYGSNISINGNVYIYSTNRNQFGIYNSRLSEVNIAQNNDIIYNSRY
QNFSISFWVRIPKHYKPMNHNREYTIINCMGNNNSGWKISLRTVRDCEIIWTLQDTSGNKEN
LIFRYEELNRISNYINKWIFVTITNNRLGNSRIYINGNLIVEKSISNLGDIHVSDNILFKIVGCDDE
TYVGIRYFKVFNTELDKTEIETLYSNEPDPSILKNYWGNYLLYNKKYYLFNLLRKDKYITLNSG
ILNINQQRGVTEGSVFLNYKLYEGVEVIIRKNGPIDISNTDNFVRKNDLAYINWDRGVEYRLY
ADTKSEKEKHRTSNLNDSLGQIIVMDSIGNNCTMNFQNNNGSNIGLLGFHSNNLVASSWYY
NNIRRNTSSNGCFWSSISKENGWKE
Botulinum G (866-1297) neuronal binding domain amino acid seguence
(SEQ ID NO: 12):
SSNAILSLSYRGGRLIDSSGYGATMNVGSDVIFNDIGNGQFKLNNSENSNITAHQSKFVVYD
SMFDNFSINFWVRTPKYNNNDIQTYLQNEYTIISCIKNDSGWKVSIKGNRIIWTLIDVNAKSKS
IFFEYSIKDNISDYINKWFSITITNDRLGNANIYINGSLKKSEKILNLDRINSSNDIDFKLINCTDT
TKFVWIKDFNIFGRELNATEVSSLYWIQSSTNTLKDFWGNPLRYDTQYYLFNQGMQNIYIKY
FSKASMGETAPRTNFNNAAINYQNLYLGLRFIIKKASNSRNINNDNIVREGDYIYLNIDNISDE
SYRVYVLVNSKEIQTQLFLAPINDDPTFYDVLQIKKYYEKTTYNCQILCEKDTKTFGLFGIGKF
VKDYGYVWDTYDNYFCISQWYLRRISENINKLRLGCNWQFIPVDEGWTE
LHn/A amino acid seguence for E.Coli expression (SEQ ID NO: 14;
Thrombin-cleaving site is underlined):
MPFVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGSTIDT
ELKVIDTNCINVIQPDGSYRSEELNLVIIGPSADIIQFECKSFGHEVLNLTRNGYGSTQYIRFSP
DFTFGFEESLEVDTNPLLGAGKFATDPAVTLAHELIHAGHRLYGIAINPNRVFKVNTNAYYE
MSGLEVSFEELRTFGGHDAKFIDSLQENEFRLYYYNKFKDIASTLNKAKSIVGTTASLQYMK
NVFKEKYLLSEDTSGKFSVDKLKFDKLYKMLTEIYTEDNFVKFFKVLNRKTYLNFDKAVFKINI
VPKVNYTIYDGFNLRNTNLAANFNGQNTEINNMNFTKLKNFTGLFEFYKLLCVRGIITSKTKS
LDKGYNKAKSLVPRGSQALNDLCIKVNNWDLFFSPSEDNFTNDLNKGEEITSDTNIEAAEEN
ISLDLIQQYYLTFNFDNEPENISIENLSSDIIGQLELMPNIERFPNGKKYELDKYTMFHYLRAQ
EFEHGKSRIALTNSVNEALLNPSRVYTFFSSDYVKKVNKATEAAMFLGWVEQLVYDFTDET
SEVSTTDKIADITIIIPYIGPALNIGNMLYKDDFVGALIFSGAVILLEFIPEIAIPVLGTFALVSYIAN
KVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIKNI
LHn/A-Spycatcher amino acid sequence (Spycatcher is in bold;
Thrombin-cleavinq site is underlined, addition caused by recombinant
fusion in italics) (SEQ ID NO: 15):
MPFVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGSTIDT
ELKVIDTNCINVIQPDGSYRSEELNLVIIGPSADIIQFECKSFGHEVLNLTRNGYGSTQYIRFSP
DFTFGFEESLEVDTNPLLGAGKFATDPAVTLAHELIHAGHRLYGIAINPNRVFKVNTNAYYE
MSGLEVSFEELRTFGGHDAKFIDSLQENEFRLYYYNKFKDIASTLNKAKSIVGTTASLQYMK
NVFKEKYLLSEDTSGKFSVDKLKFDKLYKMLTEIYTEDNFVKFFKVLNRKTYLNFDKAVFKINI
VPKVNYTIYDGFNLRNTNLAANFNGQNTEINNMNFTKLKNFTGLFEFYKLLCVRGIITSKTKS
LDKGYNKAKSLVPRGSQALNDLCIKVNNWDLFFSPSEDNFTNDLNKGEEITSDTNIEAAEEN
ISLDLIQQYYLTFNFDNEPENISIENLSSDIIGQLELMPNIERFPNGKKYELDKYTMFHYLRAQ
EFEHGKSRIALTNSVNEALLNPSRVYTFFSSDYVKKVNKATEAAMFLGWVEQLVYDFTDET
SEVSTTDKIADITIIIPYIGPALNIGNMLYKDDFVGALIFSGAVILLEFIPEIAIPVLGTFALVSYIAN
KVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIKNIRSGIL
DSMGGSGSGSGAMVDTLSGLSSEQGQSGDMTIEEDSATHIKFSKRDEDGKELAGATMEL
RDSSGKTISTWISDGQVKDFYLYPGKYTFVETAAPDGYEVATAITFTVNEQGQVTVNGKAT
KGDLE
Spytaq-Hc/A amino acid sequence (Spytag is in bold, addition
caused by recombinant fusion is in italics) (SEQ ID NO: 16):
AHIVMVDAYKPTKGSSMGRLELINTSILNLRYESNHLIDLSRYASKINIGSKVNFDPIDKNQIQ
LFNLESSKIEVILKNAIVYNSMYENFSTSFWIRIPKYFNSISLNNEYTIINCMENNSGWKVSLN
YGEIIWTLQDTQEIKQRVVFKYSQMINISDYINRWIFVTITNNRLNNSKIYINGRLIDQKPISNLG
NIHASNNIMFKLDGCRDTHRYIWIKYFNLFDKELNEKEIKDLYDNQSNSGILKDFWGDYLQY
DKPYYMLNLYDPNKYVDVNNVGIRGYMYLKGPRGSVMTTNIYLNSSLYRGTKFIIKKYASGN
KDNIVRNNDRVYINVVVKNKEYRLATNASQAGVEKILSALEIPDVGNLSQVVVMKSKNDQGI
TNKCKMNLQDNNGNDIGFIGFHQFNNIAKLVASNWYNRQIERSSRTLGCSWEFIPVDDGW
GERPL (Hc/A 874-1296 aa)
LHn/A-extension-Spycatcher amino acid sequence (Spycatcher is
underlined, the extension is in bold, additions caused by
recombinant fusion in italics) (SEQ ID NO: 17):
MPFVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGSTIDT
ELKVIDTNCINVIQPDGSYRSEELNLVIIGPSADIIQFECKSFGHEVLNLTRNGYGSTQYIRFSP
DFTFGFEESLEVDTNPLLGAGKFATDPAVTLAHELIHAGHRLYGIAINPNRVFKVNTNAYYE
MSGLEVSFEELRTFGGHDAKFIDSLQENEFRLYYYNKFKDIASTLNKAKSIVGTTASLQYMK
NVFKEKYLLSEDTSGKFSVDKLKFDKLYKMLTEIYTEDNFVKFFKVLNRKTYLNFDKAVFKINI
VPKVNYTIYDGFNLRNTNLAANFNGQNTEINNMNFTKLKNFTGLFEFYKLLCVRGIITSKTKS
LDKGYNKAKSLVPRGSQALNDLCIKVNNWDLFFSPSEDNFTNDLNKGEEITSDTNIEAAEEN
ISLDLIQQYYLTFNFDNEPENISIENLSSDIIGQLELMPNIERFPNGKKYELDKYTMFHYLRAQ
EFEHGKSRIALTNSVNEALLNPSRVYTFFSSDYVKKVNKATEAAMFLGWWEQLVYDFTDET
SEVSTTDKIADITIIIPYIGPALNIGNMLYKDDFVGALIFSGAVILLEFIPEIAIPVLGTFALVSYIAN
KVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIKNIRSGIL
DSMGKDRTQELRTAKDSDDDDDVTVTVDRDRFMDEFFEQVEEIRGFIDKIAENVEEVKRK
HSAILASPNPDEKTKEELEELMSDIKKTANKVRSKLKSIEQSIEQEEGLNRSSADLRIRKTQ
HSTLSRKFVEVMSEYNATQSDYRERCKGRIQRQLEITGRTTSMGGSGSGSGAMVDTLSGL
SSEQGQSGDMTIEEDSATHIKFSKRDEDGKELAGATMELRDSSGKTISTWISDGQVKDFYL
YPGKYTFVETAAPDGYEVATAITFTVNEQGQVTVNGKATKGDLE
Rigid trihelical domain extension amino acid sequence (SEQ ID NO: 18):
KDRTQELRTAKDSDDDDDVTVTVDRDRFMDEFFEQVEEIRGFIDKIAENVEEVKRKHSAILA
SPNPDEKTKEELEELMSDIKKTANKVRSKLKSIEQSIEQEEGLNRSSADLRIRKTQHSTLSRK
FVEVMSEYNATQSDYRERCKGRIQRQLEITGRTT
LHn/A- extension amino acid sequence (extension in bold, addition
caused by recombinant fusion is in italics) (SEQ ID NO: 19):
MPFVNKQFNYKDPVNGVDIAYIKIPNAGQMQPVKAFKIHNKIWVIPERDTFTNPEEGDLNPP
PEAKQVPVSYYDSTYLSTDNEKDNYLKGVTKLFERIYSTDLGRMLLTSIVRGIPFWGGSTIDT
ELKVIDTNCINVIQPDGSYRSEELNLVIIGPSADIIQFECKSFGHEVLNLTRNGYGSTQYIRFSP
DFTFGFEESLEVDTNPLLGAGKFATDPAVTLAHELIHAGHRLYGIAINPNRVFKVNTNAYYE
MSGLEVSFEELRTFGGHDAKFIDSLQENEFRLYYYNKFKDIASTLNKAKSIVGTTASLQYMK
NVFKEKYLLSEDTSGKFSVDKLKFDKLYKMLTEIYTEDNFVKFFKVLNRKTYLNFDKAVFKINI
VPKVNYTIYDGFNLRNTNLAANFNGQNTEINNMNFTKLKNFTGLFEFYKLLCVRGIITSKTKS
LDKGYNKAKSLVPRGSQALNDLCIKVNNWDLFFSPSEDNFTNDLNKGEEITSDTNIEAAEEN
ISLDLIQQYYLTFNFDNEPENISIENLSSDIIGQLELMPNIERFPNGKKYELDKYTMFHYLRAQ
EFEHGKSRIALTNSVNEALLNPSRVYTFFSSDYVKKVNKATEAAMFLGWVEQLVYDFTDET
SEVSTTDKIADITIIIPYIGPALNIGNMLYKDDFVGALIFSGAVILLEFIPEIAIPVLGTFALVSYIAN
KVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDA
SLKDALLKYIYDNRGTLIGQVDRLKDKVNNTLSTDIPFQLSKYVDNQRLLSTFTEYIKNIRSGIL
DSMGKDRTQELRTAKDSDDDDDVTVTVDRDRFMDEFFEQVEEIRGFIDKIAENVEEVKRK
HSAILASPNPDEKTKEELEELMSDIKKTANKVRSKLKSIEQSIEQEEGLNRSSADLRIRKTQ
HSTLSRKFVEVMSEYNATQSDYRERCKGRIQRQLEITGRTTSMGGSGSGSG
Spytag Hc/C amino acid sequence (Spytag is in bold, addition caused
by recombinant fusion is in italics) (SEQ ID NO: 20):
AHIVMVDAYKPTKGSSMGRLELNDSKILSLQNRKNTLVDTSGYNAEVSEEGDVQLNPIFPF
DFKLGSSGEDRGKVIVTQNENIVYNSMYESFSISFWIRINKWVSNLPGYTIIDSVKNNSGWSI
GIISNFLVFTLKQNEDSEQSINFSYDISNNAPGYNKWFFVTVTNNMMGNMKIYINGKLIDTIKV
KELTGINFSKTITFEINKIPDTGLITSDSDNINMWIRDFYIFAKELDGKDINILFNSLQYTNVVKD
YWGNDLRYNKEYYMVNIDYLNRYMYANSRQIVFNTRRNNNDFNEGYKIIIKRIRGNTNDTRV
RGGDILYFDMTINNKAYNLFMKNETMYADNHSTEDIYAIGLREQTKDINDNIIFQIQPMNNTY
YYASQIFKSNFNGENISGICSIGTYRFRLGGDWYRHNYLVPTVKQGNYASLLESTSTHWGF
VPVSE (Hc/C 872-1291 aa)
Additional Isopeptides pairs (cognate protein + peptide)
Pair 1: SpvCatcher002 amino acid sequence (SEQ ID NO: 21) [30]:
AMVTTLSGLSGEQGPSGDMTTEEDSATHIKFSKRDEDGRELAGATMELRDSSGKTISTWIS
DGHVKDFYLYPGKYTFVETAAPDGYEVATAITFTVNEQGQVTVNGEATKGDLET
SpyTaq002 amino acid sequence (SEQ ID NO: 22) [30]:
VPTIVMVDAYKRYK
Pair 2: Snoop Catcher amino acid sequence (SEQ ID NO: 23) [20]:
AVFSLQKQHPDYPDIYGAIDQNGTYQNVRTGEDGKLTFKNLSDGKYRLFENSEPAGYKPVQ
NKPIVAFQIVNGEVRDVTSIVPQDIPATYEFTNGKHYITNEPIPPK
SnoopTaq amino acid sequence (SEQ ID NO: 24) [20]:
KLGDIEFIKVNK
Pair 3: Sdy Catcher amino acid sequence (SEQ ID NO: 25) [21]:
SGLSGETGQSGNTTIEEDSTTHVKFSKRDANGKELAGAMIELRNLSGQTIQSWISDGTVKVF
YLMPGTYQFVETAAPEGYELAAPITFTIDEKGQIWVDS
SdyTag amino acid sequence (SEQ ID NO: 26) [21]:
DPIVMIDNDKPIT
Pair 4: Pilin-C amino acid sequence (SEQ ID NO: 27) [31]:
ATTVHGETVVNGAKLTVTKNLDLVNSNALIPNTDFTFKIEPDTTVNEDGNKFKGVALNTPMT
KVTYTNSDKGGSNTKTAEFDFSEVTFEKPGVYYYKVTEEKIDKVPGVSYDTTSYTVQVHVL
WNEEQQKPVATYIVGYKEGSKVPIQFKNSLDSTTLTVKKKVSGTGGDRSKDFNFGLTLKAN
QYYKASEKVMIEKTTKGGQAPVQTEASIDQLYHFTLKDGESIKVTNLPVGVDYVVTEDDYKS
EKYTTNVEVSPQDGAVKNIAGNSTEQETSTDKDMTI
Isopeptaq-C amino acid sequence (SEQ ID NO: 28) [31]:
TDKDMTITFTNKKDAE
Pair 5: Pilin-N amino acid sequence (SEQ ID NO: 29) [31]:
LIPNTDFTFKIEPDTTVNEDGNKFKGVALNTPMTKVTYTNSDKGGSNTKTAEFDFSEVTFEK
PGVYYYKVTEEKIDKVPGVSYDTTSYTVQVHVLWNEEQQKPVATYIVGYKEGSKVPIQFKN
SLDSTTLTVKKKVSGTGGDRSKDFNFGLTLKANQYYKASEKVMIEKTTKGGQAPVQTEASI
DQLYHFTLKDGESIKVTNLPVGVDYVVTEDDYKSEKYTTNVEVSPQDGAVKNIAGNSTEQE
TSTDKDMTITFTNKKDFE
Isopeptaq-N amino acid sequence (SEQ ID NO: 30) [31]:
ATTVHGETVVNGAKLTVTKNLDLVNSNA
LHn/A-2xSpycatcher amino acid sequence (SEQ ID NO: 31):
(The first and second Spycatchers are in bold, additional
amino acids caused by recombinant fusion, i.e. restriction
sites, are underlined, thrombin cleavaqe site is in italics
and underlined)
PGMPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGGSTI
DTELKVIDTNCINVIQPDGSYRSEELNLVIIGPSADIIQFECKSFGHEVLNLTRNGYGSTQYIRF
SPDFTFGFEESLEVDTNPLLGAGKFATDPAVTLAHELIHAGHRLYGIAINPNRVFKVNTNAYY
EMSGLEVSFEELRTFGGHDAKFIDSLQENEFRLYYYNKFKDIASTLNKAKSIVGTTASLQYM
KNVFKEKYLLSEDTSGKFSVDKLKFDKLYKMLTEIYTEDNFVKFFKVLNRKTYLNFDKAVFKI
NIVPKVNYTIYDGFNLRNTNLAANFNGQNTEINNMNFTKLKNFTGLFEFYKLLCVRGIITSKTK
SLDKGYNKAKSLVPRGSQALNDLCIKVNNWDLFFSPSEDNFTNDLNKGEEITSDTNIEAAEE
NISLDLIQQYYLTFNFDNEPENISIENLSSDIIGQLELMPNIERFPNGKKYELDKYTMFHYLRA
QEFEHGKSRIALTNSVNEALLNPSRVYTFFSSDYVKKVNKATEAAMFLGWVEQLVYDFTDE
TSEVSTTDKIADITIHPYIGPALNIGNMLYKDDFVGALIFSGAVILLEFIPEIAIPVLGTFALVSYIA
NKVLTVQTIDNALSKRNEKWDEVYKYIVTNWLAKVNTQIDLIRKKMKEALENQAEATKAIINY
QYNQYTEEEKNNINFNIDDLSSKLNESINKAMININKFLNQCSVSYLMNSMIPYGVKRLEDFD
ASLKDALLKYIYDNRGTLIGQVDRLKDKVNNTLSTDIPFQLSKYVDNQRLLSTFTEYIKNIRSG
ILDSMGGSGSGSGAMVDTLSGLSSEQGQSGDMTIEEDSATHIKFSKRDEDGKELAGATM
ELRDSSGKTISTWISDGQVKDFYLYPGKYTFVETAAPDGYEVATAITFTVNEQGQVTVNGK
ATKGDLEGASGGGGASSAGGAMVDTLSGLSSEQGQSGDMTIEEDSATHIKFSKRDEDGK
ELAGATMELRDSSGKTISTWISDGQVKDFYLYPGKYTFVETAAPDGYEVATAITFTVNEQG
QVTVNGKATKGDLELKLNSS

### GENERAL REFERENCES

1. Foran P G, Davletov B, Meunier F A. Getting muscles moving again after botulinum toxin: novel therapeutic challenges. Trends in molecular medicine. 2003; 9(7):291-9.
2. Rummel A, Binz, T. Botulinum Neurotoxins: Springer; Current Topics in Microbiology and Immunology, 2013.
3. Rossetto O, Pirazzini M, Montecucco C. Botulinum neurotoxins: genetic, structural and mechanistic insights. Nat Rev Microbiol. 2014; 12(8):535-49.
4. Pirazzini M, Rossetto O, Eleopra R, Montecucco C. Botulinum Neurotoxins: Biology, Pharmacology, and Toxicology. Pharmacol Rev. 2017; 69(2):200-35.
5. Aoki K R, Guyer B. Botulinum toxin type A and other botulinum toxin serotypes: a comparative review of biochemical and pharmacological actions. European journal of neurology. 2001; 8 Suppl 5:21-9.
6. Davletov B, Bajohrs M, Binz T. Beyond BOTOX: advantages and limitations of individual botulinum neurotoxins. Trends in neurosciences. 2005; 28(8):446-52.
7. Dolly O. Synaptic transmission: inhibition of neurotransmitter release by botulinum toxins. Headache. 2003; 43 Suppl 1:S16-24.
8. Jankovic J. Botulinum toxin in clinical practice. Journal of neurology, neurosurgery, and psychiatry. 2004; 75(7):951-7.
9. Johnson E A. Clostridial toxins as therapeutic agents: benefits of nature's most toxic proteins. Annual review of microbiology. 1999; 53:551-75.
10. Montecucco C, Schiavo G. Tetanus and botulism neurotoxins: a new group of zinc proteases. Trends in biochemical sciences. 1993; 18(9):324-7.
11. Simpson L L. Identification of the major steps in botulinum toxin action. Annual review of pharmacology and toxicology. 2004; 44:167-93.
12. Binz T, Kurazono H, Wine M, Frevert J, Wernars K, Niemann H. The complete sequence of botulinum neurotoxin type A and comparison with other clostridial neurotoxins. The Journal of biological chemistry. 1990; 265(16):9153-8.
13. Ferrari E, Maywood E S, Restani L, et al. Re-assembled botulinum neurotoxin inhibits CNS functions without systemic toxicity. Toxins. 2011; 3(4):345-55.
14. Darios F, Niranjan D, Ferrari E, et al. SNARE tagging allows stepwise assembly of a multimodular medicinal toxin. Proc Natl Acad Sci USA. 2010; 107(42):18197-201.
15. Mangione A S, Obara I, Maiaru M, et al. Nonparalytic botulinum molecules for the control of pain. Pain. 2016; 157(5):1045-55.
16. Zhang S, Masuyer G, Zhang J, et al. Identification and characterization of a novel botulinum neurotoxin. Nat Commun. 2017; 8:14130.
17. Leese C, Bresnahan R, Doran C, et al. Duplication of clostridial binding domains for enhanced macromolecular delivery into neurons. Toxicon: X. 2020; 5:100019.
18. Bade S, Rummel A, Reisinger C, et al. Botulinum neurotoxin type D enables cytosolic delivery of enzymatically active cargo proteins to neurones via unfolded translocation intermediates. J Neurochem. 2004; 91(6):1461-72.
19. Zakeri B, Fierer J O, Celik E, et al. Peptide tag forming a rapid covalent bond to a protein, through engineering a bacterial adhesin. Proc Natl Acad Sci USA. 2012; 109(12):E690-7.
20. Veggiani G, Nakamura T, Brenner M D, et al. Programmable polyproteams built using twin peptide superglues. P Natl Acad Sci USA. 2016; 113(5):1202-7.
21. Tan L L, Hoon S S, Wong F T. Kinetic Controlled Tag-Catcher Interactions for Directed Covalent Protein Assembly. Plos One. 2016; 11(10).
22. Arsenault J, Ferrari E, Niranjan D, et al. Stapling of the botulinum type A protease to growth factors and neuropeptides allows selective targeting of neuroendocrine cells. J Neurochem. 2013; 126(2):223-33.
23. Ma H, Meng J, Wang J, Hearty S, Dolly J O, O'Kennedy R. Targeted delivery of a SNARE protease to sensory neurons using a single chain antibody (scFv) against the extracellular domain of P2X(3) inhibits the release of a pain mediator. The Biochemical journal. 2014; 462(2):247-56.
24. Vauquelin G, Charlton S J. Exploring avidity: understanding the potential gains in functional affinity and target residence time of bivalent and heterobivalent ligands. Br J Pharmacol. 2013; 168(8):1771-85.
25. Ashton A C, Dolly J O. Characterization of the inhibitory action of botulinum neurotoxin type A on the release of several transmitters from rat cerebrocortical synaptosomes. J Neurochem. 1988; 50(6):1808-16.
26. Capogna M, McKinney R A, O'Connor V, Gahwiler B H, Thompson S M. Ca2+ or Sr2+ partially rescues synaptic transmission in hippocampal cultures treated with botulinum toxin A and C, but not tetanus toxin. The Journal of neuroscience: the official journal of the Society for Neuroscience. 1997; 17(19):7190-202.
27. Costantin L, Bozzi Y, Richichi C, et al. Antiepileptic effects of botulinum neurotoxin E. The Journal of neuroscience: the official journal of the Society for Neuroscience. 2005; 25(8):1943-51.
28. Luvisetto S, Marinelli S, Rossetto O, Montecucco C, Pavone F. Central injection of botulinum neurotoxins: behavioural effects in mice. Behavioural pharmacology. 2004; 15(3):233-40.
29. Fernandez I, Ubach J, Dulubova I, Zhang X, Sudhof T C, Rizo J. Three-dimensional structure of an evolutionarily conserved N-terminal domain of syntaxin 1A. Cell. 1998; 94(6):841-9.
30. Keeble A H, Banerjee A, Ferla M P, Reddington S C, Anuar I N A K, Howarth M. Evolving Accelerated Amidation by SpyTag/SpyCatcher to Analyze Membrane Dynamics. Angew Chem Int Edit. 2017; 56(52):16521-5.
31. Kang H J, Coulibaly F, Clow F, Proft T, Baker E N. Stabilizing isopeptide bonds revealed in gram-positive bacterial pilus structure. Science. 2007; 318(5856):1625-8.

Table of abbreviations

| Abbreviation | Meaning |
|---|---|
| aa | Amino acids |
| BonBot | Bonded botulinum neurotoxin |
| Bot | Botulinum Neurotoxin |
| ext | Extended |
| ext-BonBot | Extended bonded botulinum neurotoxin |
| H | Heavy Chain |
| Hc | Heavy Chain C-terminal part |
| Hn | Heavy chain N-terminal part |
| kDa | Kilodalton |
| L | Light Chain |
| LHn | Light Chain Heavy chain N-terminal part |
| Nbd | Neuronal binding domain |
| ng | Nanogram |
| nm | Nanometers |
| SEQ | Sequence |
| SNAP25 | Synaptosome-associated protein of 25 kDa |
| SNAREs | Soluble N-ethylmaleimide-sensitive fusion protein |
| | attachment receptors |
| VAMP | Vesicle Associated Membrane Protein |

1. A neurotoxin comprising a SNARE peptidase domain, a translocation domain and a neuronal binding domain, wherein two of the domains are present within a disulphide-linked polypeptide that is covalently linked to the third domain via an isopeptide bond.

2. The neurotoxin of claim 1, wherein the SNARE peptidase domain, translocation domain and neuronal binding domain are botulinum neurotoxin SNARE peptidase, translocation and neuronal binding domains.

3. The neurotoxin of claim 2, wherein the SNARE peptidase domain, translocation domain and neuronal binding domain are botulinum neurotoxin type A SNARE peptidase, translocation and neuronal binding domain.

4. The neurotoxin of claim 1, wherein the SNARE peptidase domain and the translocation domain are present within the disulphide-linked polypeptide that is covalently linked to the neuronal binding domain via an isopeptide bond, optionally wherein the disulphide-linked polypeptide further comprises a rigid trihelical extension.

5. The neurotoxin of claim 1, wherein the isopeptide bond is formed between a peptide and its cognate protein, wherein:
a) the peptide is attached to the disulphide-linked polypeptide and the cognate protein is attached to the third domain; or
b) the cognate protein attached to the disulphide-linked polypeptide and the peptide is attached to the third domain.

6. The neurotoxin of claim 5, wherein the peptide comprises an amino acid sequence of SEQ ID NO:1 and the cognate protein comprises an amino acid sequence of SEQ ID NO:2.

7. The neurotoxin of claim 1, wherein the neurotoxin comprises:
a) a disulphide-linked polypeptide comprising the SNARE peptidase domain, the translocation domain and a C-terminal cognate protein comprising the amino acid sequence of SEQ ID NO: 2; and
b) the neuronal binding domain attached to an N-terminal peptide comprising the amino acid sequence of SEQ ID NO:1,
wherein the disulphide-linked polypeptide is covalently linked to the neuronal binding domain via an isopeptide bond between the cognate protein of a) and peptide of b).

8. The neurotoxin of claim 7, wherein the SNARE peptidase domain comprises an amino acid sequence having at least 80% identity to the amino acid sequence of SEQ ID NO: 3 and the translocation domain comprises the amino acid sequence of SEQ ID NO:4 or SEQ ID NO: 5.

9. The neurotoxin of claim 8, wherein the neuronal binding domain comprises the amino acid sequence of SEQ ID NO: 6 or SEQ ID NO:7.

10. A composition comprising the neurotoxin of claim 1, and a pharmaceutically acceptable excipient, adjuvant, diluent or carrier.

11.-14. (canceled)

15. A therapeutic method, comprising administering the composition of claim 10 to a subject in need thereof.

16. A method of preventing, regulating, or reducing neuropathic pain or sweating in a subject, comprising administering the composition of claim 10 to the subject.

17. A method of producing a neurotoxin comprising a SNARE peptidase domain, a translocation domain and a neuronal binding domain, the method comprising mixing a disulphide-linked polypeptide comprising two of the domains with a polypeptide comprising the third domain, wherein
(i) a peptide is attached to the disulphide-linked polypeptide and a cognate protein is attached to the third domain; or
(ii) a cognate protein attached to the disulphide-linked polypeptide and a peptide is attached to the third domain,
such that, on mixing, an isopeptide bond is formed between the peptide and its cognate protein to covalently link the disulphide-linked polypeptide to the third domain.

18. A kit for producing a neurotoxin comprising a SNARE peptidase domain, a translocation domain and a neuronal binding domain, the kit comprising:
(a) a disulphide-linked polypeptide comprising two of the domains; and
(b) a polypeptide comprising the third domain, wherein
(i) a peptide is attached to the disulphide-linked polypeptide and a cognate protein is attached to the third domain; or
(ii) a cognate protein attached to the disulphide-linked polypeptide and a peptide is attached to the third domain,
such that, on mixing, an isopeptide bond is formed between the peptide and its cognate protein to covalently link the disulphide-linked polypeptide to the third domain.

19. The method of claim 17, wherein the SNARE peptidase domain, translocation domain and neuronal binding domain are botulinum neurotoxin SNARE peptidase, translocation and neuronal binding domains.

20. The method of claim 19, wherein the SNARE peptidase domain, translocation domain and neuronal binding domain are botulinum neurotoxin type A SNARE peptidase, translocation and neuronal binding domain.

21. The method of claim 17, wherein the peptide comprises an amino acid sequence of SEQ ID NO:1 and the cognate protein comprises an amino acid sequence of SEQ ID NO:2.

22. The method of claim 21, wherein:
a) the disulphide-linked polypeptide comprises the SNARE peptidase domain, the translocation domain and a C-terminal cognate protein comprising the amino acid sequence of SEQ ID NO: 2; and
b) the polypeptide comprising the third domain comprises the neuronal binding domain attached to an N-terminal peptide comprising the amino acid sequence of SEQ ID NO:1,
wherein the disulphide-linked polypeptide is covalently linked to the neuronal binding domain via an isopeptide bond between the cognate protein of a) and peptide of b), and optionally wherein the disulphide-linked polypeptide further comprises a rigid trihelical extension.

23. The method of claim 22, wherein the SNARE peptidase domain comprises an amino acid sequence having at least 80% identity to the amino acid sequence of SEQ ID NO: 3 and the translocation domain comprises the amino acid sequence of SEQ ID NO:4 or SEQ ID NO: 5.

24. The method of claim 23, wherein the neuronal binding domain comprises the amino acid sequence of SEQ ID NO: 6 or SEQ ID NO:7.


SEQUENCE LISTING (31 sequences)

SEQ ID NO: 1 - protein, 11 aa, Artificial Sequence
  Spytag
AHIVMVDAYKP

SEQ ID NO: 2 - protein, 114 aa, Artificial Sequence
  Spycatcher
AMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDLE

SEQ ID NO: 3 - protein, 448 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 4 - protein, 426 aa, Clostridium botulinum
QALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNI

SEQ ID NO: 5 - protein, 727 aa, Artificial Sequence
  Ext-Bot/A translocation domain amino acid sequence for E. coli expression
AKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIRSGILDSMGKDRTQELRTAKDSDDDDDVTVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTSMGGSGSGSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDLE

SEQ ID NO: 6 - protein, 423 aa, Clostridium botulinum
INTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 7 - protein, 423 aa, Clostridium botulinum
NDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 8 - protein, 430 aa, Clostridium botulinum
NNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 9 - protein, 412 aa, Clostridium botulinum
NDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 10 - protein, 402 aa, Clostridium botulinum
SSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQE

SEQ ID NO: 11 - protein, 409 aa, Clostridium botulinum
KDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 12 - protein, 432 aa, Clostridium botulinum
SSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 13 - protein, 6 aa, Clostridium botulinum
LVPRGS

SEQ ID NO: 14 - protein, 883 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKAKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNI

SEQ ID NO: 15 - protein, 1013 aa, Artificial Sequence
  LHn/A-Spycatcher
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKAKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIRSGILDSMGGSGSGSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDLE

SEQ ID NO: 16 - protein, 445 aa, Artificial Sequence
  Spytag-Hc/A
AHIVMVDAYKPTKGSSMGRLELINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 17 - protein, 1175 aa, Artificial Sequence
  LHn/A-extension-Spycatcher
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKAKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIRSGILDSMGKDRTQELRTAKDSDDDDDVTVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTSMGGSGSGSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDLE

SEQ ID NO: 18 - protein, 159 aa, Artificial Sequence
  Rigid trihelical domain extension
KDRTQELRTAKDSDDDDDVTVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTT

SEQ ID NO: 19 - protein, 1061 aa, Artificial Sequence
  LHn/A- extension
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKAKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIRSGILDSMGKDRTQELRTAKDSDDDDDVTVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTSMGGSGSGSG

SEQ ID NO: 20 - protein, 445 aa, Artificial Sequence
  Spytag Hc/C
AHIVMVDAYKPTKGSSMGRLELNDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 21 - protein, 115 aa, Artificial Sequence
  SpyCatcher002
AMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDLET

SEQ ID NO: 22 - protein, 14 aa, Artificial Sequence
  SpyTag002
VPTIVMVDAYKRYK

SEQ ID NO: 23 - protein, 107 aa, Artificial Sequence
  Snoop Catcher
AVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 24 - protein, 12 aa, Artificial Sequence
  SnoopTag
KLGDIEFIKVNK

SEQ ID NO: 25 - protein, 100 aa, Artificial Sequence
  Sdy Catcher
SGLSGETGQSGNTTIEEDSTTHVKFSKRDANGKELAGAMIELRNLSGQTIQSWISDGTVKVFYLMPGTYQFVETAAPEGYELAAPITFTIDEKGQIWVDS

SEQ ID NO: 26 - protein, 13 aa, Artificial Sequence
  SdyTag
DPIVMIDNDKPIT

SEQ ID NO: 27 - protein, 282 aa, Artificial Sequence
  Pilin-C
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 28 - protein, 16 aa, Artificial Sequence
  Isopeptag-C
TDKDMTITFTNKKDAE

SEQ ID NO: 29 - protein, 263 aa, Artificial Sequence
  Pilin-N
LIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTITFTNKKDFE

SEQ ID NO: 30 - protein, 28 aa, Artificial Sequence
  Isopeptag-N
ATTVHGETVVNGAKLTVTKNLDLVNSNA

SEQ ID NO: 31 - protein, 1148 aa, Artificial Sequence
  LHn/A-2xSpycatcher
PGMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKAKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIRSGILDSMGGSGSGSGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDLEGASGGGGASSAGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDLELKLNSS